(19) United States
(12) Patent Application Publication
(10) Pub. No.: US 2006/0251675 A1
(43) Pub. Date: Nov. 9, 2006

(54) Mutant cholera holotoxin as an adjuvant and an antigen carrier protein

(76) Inventor: Michael Hagen, Pittsford, NY (US)

Correspondence Address: WYETH;PATENT LAW GROUP, 5 GIRALDA FARMS, MADISON, NJ, 07940, US

(21) Appl. No.: 10/549,302

(22) PCT Filed: Mar. 11, 2004
(86) PCT No.: PCT/US04/07673

§ 371 Date: Sep. 15, 2005

Related U.S. Application Data
(60) Provisional application No. 60/455,214, filed on Mar. 17, 2003.

(51) Int. Cl. A61K 39/106; A61K 39/116; A61K 38/20; C07K 14/28; A61K 38/19; A61K 31/739; A61K 31/7008

(52) U.S. Cl. 424/203.1; 530/350; 424/261.1; 424/85.2; 424/85.1; 514/54; 514/62

(57) ABSTRACT

The invention relates to a mutant cholera holotoxin having reduced toxicity, which functions both as an adjuvant and an antigen carrier. In a particular embodiment, the cholera holotoxin is genetically modified at least at amino acid residue 29 of the A subunit, wherein the genetic modification comprises an amino acid substitution of the wild-type glutamic acid at position 29, wherein the substitution is not an aspartic acid.

### FIELD OF THE INVENTION

The present invention relates to a mutant cholera holotoxin as an adjuvant and an antigen carrier, wherein the mutant cholera holotoxin has reduced toxicity compared to a wild-type cholera holotoxin. More particularly, the cholera holotoxin protein is genetically modified at least at amino acid residue 29 of the A subunit, wherein the genetic modification comprises an amino acid substitution of the wild-type glutamic acid at position 29, wherein the substitution is not an aspartic acid.

### BACKGROUND OF THE INVENTION

The immune system uses a variety of mechanisms for resisting, attacking and clearing pathogens. However, not all of these mechanisms are necessarily activated after immunization. Protective immunity induced by immunization is dependent on the capacity of the antigen to elicit the appropriate immune response to either resist or eliminate the pathogen. Depending on the pathogen, this may require a cell-mediated and/or a humoral immune response.

Among the strategies investigated to elicit an immune response is the use of mucosal adjuvants. It is known that cholera toxin (CT) is one of the most potent adjuvants, and that the co-administration of CT with an unrelated antigen results in the induction of concurrent circulating and mucosal antibody responses to that antigen (Elson and Ealding, 1984). However, due to the inherent toxicity associated with CT, the concentration of a CT adjuvant which can be administered may reduce adjuvant activity or effect. To overcome the toxicity associated with CT adjuvants, genetic modifications have been identified which result in significant reductions in CT enzymatic activity, without a loss of its immunogenic properties (e.g., see U.S. Pat. No. 6,149,919; U.S. Pat. No. 5,874,287). In addition to CT, genetic modifications of other toxoids such as Escherichia coli heat labile toxin (LT) and pertussis toxin (PT) have been described (International Applications WO 98/42375, WO 97/02348, WO 93/13202 and WO 92/19265). Similar to CT, these mutations reduce toxicity of LT and PT, without a loss of their immunogenic properties.

A second approach to overcome problems associated with the mucosal and/or parenteral immune response(s) has been the use of antigen carrier proteins. For example, when T-independent pneumococcal polysaccharide antigens or peptide antigens are chemically conjugated to carrier proteins, enhanced immunogenicity is observed, with a booster response indicative of the formation of immunological memory (Henriksen et al., 1997). Importantly, the presence of the carrier protein in the conjugate ensures the involvement of T-helper cells in the activation of B lymphocytes and thus a qualitatively different, and improved, immune response including memory formation (de Valesco et al., 1995). Thus, antigen carrier proteins allow the conversion of poorly immunogenic antigens like polysaccharides and small peptides, to T-dependent epitopes that will elicit an immunoglobulin G (IgG) immune response following priming with the antigen and an anamnestic response on reimmunization. Additionally, for the same reasons, conjugate vaccines benefit elderly and young populations, which typically do not respond well to immunization, because of their immature or diminished immune systems.

However, the conjugation of an antigen to an antigen carrier protein (i.e., a conjugate vaccine) does not always yield an effective or desirable immune response. For example, Klipstein et al. described conjugating the E. coli heat-stable (ST) toxin to an LT carrier protein with a carbodiimde conjugating reagent, wherein the ST-LT conjugate had diminished antigenicity and increased toxicity (Klipstein et al., 1983). In fact, Klipstein reported a “critical” amount of carbodiimide reagent was necessary for conjugating the maximum amount of ST to LT, the proportion of ST present in the final conjugate is dependent on initial molar ratio of ST mixed with LT, and increasing the ratio of carbodiimide to either toxin in the conjugate resulted in a progressive decline in antigenicity and an increase in toxicity of the ST-LT conjugate.

It is therefore highly desirable to identify a cholera holotoxin having reduced toxicity, which functions both as an adjuvant and an antigen carrier. It is contemplated that the identification of such compositions, in addition to their reduced toxicity and enhanced immunogenicity, will simplify immunogen formulation, as such a compositions will function as both antigen carrier and adjuvant.

### SUMMARY OF THE INVENTION

The present invention broadly relates to a mutant cholera holotoxin, which functions as both an immune adjuvant and an antigen carrier, wherein the mutant cholera holotoxin has reduced toxicity compared to a wild-type cholera holotoxin. More particularly, the cholera holotoxin is genetically modified at least at amino acid residue 29 of the A subunit, wherein the genetic modification comprises an amino acid substitution of the wild-type glutamic acid at position 29, wherein the substitution at position 29 is not an aspartic acid.

Thus, in particular embodiments, the invention is directed to an immunogenic composition comprising a cholera holotoxin (CT) and an antigen covalently associated with the CT, wherein the CT comprises an A subunit (CT-A ) having a mutation (substitution) of at least amino acid residue 29 of SEQ ID NO:2, wherein the mutation of amino acid 29 is not an aspartic acid, wherein the CT increases immunogenicity of the antigen. In a particular embodiment, the CT is further defined as having reduced toxicity relative to a CT comprising a wild-type CT-A. In certain embodiments, the CT-A is encoded by a polynucleotide comprising a nucleic acid sequence of SEQ ID NO:1 or a degenerate variant thereof, wherein the nucleotide sequence has a genetic modification of at least codon 29 of SEQ ID NO:1. In another embodiment, amino acid residue 29 of SEQ ID NO:2 is an amino acid selected from the group consisting of Ala, Cys, Phe, Gly, His, Ile, Lys, Leu, Met, Asn, Pro, Gln, Arg, Ser, Thr, Val, Trp and Tyr. In a preferred embodiment, amino acid residue 29 of SEQ ID NO:2 is a His residue. In certain other embodiments, the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

In yet another embodiment, the immunogenic composition further comprises one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate. In still other embodiments, the immunogenic composition further comprises one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate. In certain embodiments, the composition further comprises one or more adjuvants selected from the group consisting of GM-CSF, 529SE or 529AF, QS21, IL-12, aluminum phosphate, aluminum hydroxide, Mycobacterium tuberculosis, Bordetella pertussis, bacterial lipopolysaccharides, aminoalkyl glucosamine phosphate compounds, MPL™ (3-O-deacylated monophosphoryl lipid A), a polypeptide, Quil A, STIMULON™, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor α and tumor necrosis factor β. In yet another embodiment, the composition further comprises a pharmaceutically acceptable carrier.

In another embodiment, the invention is directed to an immunogenic composition comprising a CT and an antigen covalently associated with the CT, wherein the CT comprises one or more mutations (substitutions) in the CT-A, wherein the CT increases immunogenicity of the antigen. In a particular embodiment, the CT is further defined as having reduced toxicity relative to a CT comprising a wild-type CT-A. In another embodiment, the CT-A comprises an amino acid sequence of SEQ ID NO:2. In yet another embodiment, the CT-A is encoded by a polynucleotide comprising a nucleic acid sequence of SEQ ID NO:1 or a degenerate variant thereof. In preferred embodiments, the one or more mutations are selected from the group consisting of Arg-7, Asp-9, Arg-11, Ile-16, Arg-25, Glu-29, Tyr-30, His-44, Val-53, Ser-63, Ser-68, His-70, Val-72, Val-97, Tyr-104, Pro-106, Ser-109, Glu-112 and Arg-192. In yet another preferred embodiment, one or more mutations of CT-A is at amino acid Glu-29. In a most preferred embodiment, Glu-29 is mutated to a His-29 residue. In another preferred embodiment, one or more mutations of CT-A is a double mutation at amino acids Ile-16 and Ser-68 or a double mutation at amino acids Ser-68 and Val-72. In still other embodiments, a CT-A comprises an insertion of a single amino acid in the CT-A polypeptide sequence, wherein the amino acid insertion is at amino acid position 49 of the CT-A, thereby shifting the amino acid residues originally located at positions 49, 50, etc., to positions 50, 51, etc. In a preferred embodiment, a histidine amino acid is inserted at amino acid position 49 (His-49) of the CT-A. In still other embodiments, a CT-A comprises an insertion of a two amino acids in the CT-A polypeptide sequence, wherein the amino acid insertions are at amino acid positions 35 and 36 of the CT-A, thereby shifting the original amino acid residues at positions 35 and 36 to positions 37, 38, etc. In a preferred embodiment, the amino acid inserted at position 35 is a glycine (Gly-35) and the amino acid inserted at position 36 is a proline (Pro-36). In yet another embodiment, a CT-A comprises an amino acid mutation (substitution) at position Tyr-30 of the CT-A polypeptide sequence and an insertion of two amino acids at position 31 and 32 in the CT-A polypeptide sequence, thereby shifting the original amino acid residues at positions 31 and 32 to positions 33 and 34, etc. In a preferred embodiment, the amino acid mutation at position 30 is a tryptophan (Trp-30) and the amino acid insertion at positions 31 and 32 is an alanine (Ala-31) and a histidine (His-32).

In other embodiments, the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

In further embodiments, the immunogenic composition further comprises one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate. In still another embodiment, the immunogenic composition further comprises one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate. In certain embodiments, the composition further comprises one or more adjuvants selected from the group consisting of GM-CSF, 529SE or 529AF, QS21, IL-12, aluminum phosphate, aluminum hydroxide, Mycobacterium tuberculosis, Bordetella pertussis, bacterial lipopolysaccharides, aminoalkyl glucosamine phosphate compounds, MPL™ (3-O-deacylated monophosphoryl lipid A), a polypeptide, Quil A, STIMULON™, a pertussis toxin (PT), an E. coil heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor α and tumor necrosis factor β. In other embodiments, the composition further comprises a pharmaceutically acceptable carrier.

In other embodiments, the invention is directed to an immunogenic composition comprising an Escherichia coli heat labile toxin (LT) and an antigen covalently associated with the LT, wherein the LT increases immunogenicity of the antigen. In certain embodiments, the LT is further defined as having one or more mutations in the LT-A subunit. In certain other embodiments, the one or more mutations in the LT-A subunit are selected from the group consisting of Val-53, Ser-63, Ala-72, Val-97, Tyr-104, Pro-106 and Arg-192. In yet another embodiment, the invention is directed to an immunogenic composition comprising a pertussis toxin (PT) and an antigen covalently associated with the PT, wherein the PT increases immunogenicity of the antigen. In preferred embodiments, the LT or the PT is a genetically modified LT or PT polypeptide having reduced toxicity relative to a wild-type LT or PT polypeptide. In other embodiments, the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate. In yet other embodiments, the immunogenic LT or PT composition further comprises one or more adjuvants, wherein the one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1α, IL-2, IL-4, IL-5, IL-6, IL-7, IL-8, IL-10, IL-13, IL-14, IL-15, IL-16, IL-7, IL-18, interferon-β, interferon-γ, granulocyte colony stimulating factor, tumor necrosis factor a and tumor necrosis factor β. In yet another embodiment, the immunogenic composition further comprises a pharmaceutically acceptable carrier.

In other embodiments, the invention is directed to methods of immunizing a mammalian host, the method comprising administering to the host an immunogenic amount of a composition comprising a cholera holotoxin (CT) and an antigen covalently associated with the CT, wherein the CT comprises an A subunit (CT-A) having a mutation of at least amino acid residue 29 of SEQ ID NO:2, wherein the mutation is not an aspartic acid, wherein the CT increases immunogenicity of the antigen. In certain embodiments, the invention is directed to methods of immunizing a mammalian host comprising administering to the host an immunogenic amount of a composition comprising an Escherichia coli heat labile toxin (LT) and an antigen covalently associated with the LT, wherein the LT increases immunogenicity of the antigen. In yet other embodiments, the invention is directed to methods of immunizing a mammalian host comprising administering to the host an immunogenic amount of a composition comprising a pertussis toxin (PT) and an antigen covalently associated with the PT, wherein the PT increases immunogenicity of the antigen.

Other features and advantages of the invention will be apparent from the following detailed description, from the preferred embodiments thereof, and from the claims.

### BRIEF DESCRIPTION OF THE FIGURES

FIG. 1 shows the effectiveness of CT E29H as a carrier for peptides as determined by peptide specific IgG antibody titers. Groups of 5 Swiss Webster female mice were immunized with 5 ug (total protein) of the indicated conjugates, 30 ug of Aβ 1-42 peptide, 10 ug GMCSF, 5 ug CT E29H, or 25 ug 529SE as indicated. Mice were immunized subcutaneously on week 0 and week 3. Individual sera were collected and measured for peptide specific IgG antibody titers prior to immunization, the day prior to the second immunization, and two weeks thereafter. The data represent anti-Aβ 1-42 peptide specific IgG endpoint titer Geometric means±standard error for all individual animals in the groups. Pre-immunization titers were below the level of detection at a 1/50 dilution of serum.

FIG. 2 shows the effectiveness of CT E29H as a carrier for peptides as determined by IgG subclass titers. Groups of 5 Swiss Webster female mice were immunized with 5 ug (total protein) of the indicated conjugates, 30 ug of Aβ 1-42 peptide, 10 ug GMCSF, 5 ug CT E29H, or 25 ug 529SE as indicated. Mice were immunized subcutaneously on week 0 and week 3. Individual sera were collected and measured for peptide specific IgG subclass antibody titers 2 weeks after the second immunization. The data represent anti-Aβ 1-42 peptide specific IgG1, IgG2a and IgG2b endpoint titer Geometric means±standard error for all individual animals in the groups. Pre-immunization titers were below the level of detection at a 1/50 dilution of serum.

FIG. 3 shows the effectiveness of CT E29H as a carrier for peptides in the presence or absence of 529SE as determined by IgG titers. Groups of 10 Swiss Webster female mice were immunized with 5 ug (total protein) of the indicated conjugates, with or without 25 ug 529SE as indicated. Mice were immunized subcutaneously on week 0 and week 3. Individual sera were collected and measured for peptide specific IgG antibody titers prior to immunization, the day prior to the second immunization, and two weeks thereafter. The data represent anti-Aβ 1-42 peptide specific IgG endpoint titer Geometric means±standard error for all individual animals in the groups. Pre-immunization titers were below the level of detection at a 1/50 dilution of serum.

FIG. 4 shows the effectiveness of CT E29H as a carrier for peptides in the presence or absence of 529SE as determined by IgG subclass titers. Groups of 10 Swiss Webster female mice were immunized with 5 ug (total protein) of the indicated conjugates, with or without 25 ug 529SE as indicated. Mice were immunized subcutaneously on weeks 0 and 3. Individual sera were collected and measured for peptide specific IgG subclass antibody titers 2 weeks after the second immunization (week 5). The data represent anti-Aβ 1-42 peptide specific IgG subclass endpoint titer Geometric means±standard error for all individual animals in the groups. Pre-immunization titers were below the level of detection at a 1/50 dilution of serum.

FIG. 5 shows anti-peptide IgG titers in Balb/c mice immunized with Aβ1-7 conjugates to CRM₁₉₇ or CT E29H. Groups of 5 Balb/c female mice were immunized with 5 ug of the indicated conjugate, with or without the addition of 1 ug non-conjugated CT E29H. Mice were immunized subcutaneously twice, 4 weeks apart, and bled one day prior to each immunization, and 2 weeks after the second immunization. Sera were collected for peptide-specific antibody endpoint titer determination using ELISA.

FIG. 6 shows the effect of Aβ 1-7/CT E29H conjugate dose on anti-Aβ 1-42 endpoint titers in young and old Swiss Webster mice. Groups of 10 female mice were immunized via intranasal delivery of either 5 ug Aβ 1-7/CRM₁₉₇ conjugate, or 1 ug, 5 ug or 10 ug of Aβ 1-7/CT E29H conjugate, or 5 ug Aβ 1-7/CRM₁₉₇ conjugate adjuvanted with 1 ug CT E29H. Mice received 3 immunizations 2 weeks apart, and were bled at the indicated time points the day prior to immunization.

FIG. 7A shows titers measured from pools of sera collected at 4 weeks, 8 weeks and 10 weeks.

FIG. 7B shows anti-PGM7232 titers as measured from sera collected at 10 weeks.

FIG. 8 shows titers from mice after 3 immunizations with GBS/E29H conjugate, GBS/CRM₁₉₇ conjugate or GBS/CRM₁₉₇ conjugate adjuvanted with CT E29H.

FIG. 9 demonstrates the effectiveness of CT E29H as an adjuvant and antigen carrier in the absence of exogenous adjuvant.

FIG. 10 demonstrates that CT E29H is an effective adjuvant for non-conjugated (i.e., admixed) antigens.

### DETAILED DESCRIPTION OF THE INVENTION

The invention described hereinafter, addresses the need for effective immune system adjuvants having reduced or minimal toxicity, which also function as antigen carriers (i.e., present or deliver one or more antigens to the immune system). Thus, in certain embodiments, the invention is directed to immunogenic compositions and methods of immunization comprising a mutant cholera holotoxin (hereinafter, mutant CT) as an antigen carrier protein, wherein the mutant CT antigen carrier has intrinsic adjuvant activity and reduced toxicity compared to a wild-type cholera holotoxin (hereinafter, wild-type CT). In certain other embodiments, the invention is directed to compositions and methods of immunization comprising a mutant CT as an immune adjuvant, wherein the mutant CT adjuvant has reduced toxicity compared to a wild-type CT. In still other embodiments, the invention is directed to an E. coli heat labile toxin (LT) or a pertussis toxin (PT) as an antigen carrier protein and an immune adjuvant. In a preferred embodiment, the LT or PT is a mutant LT or mutant PT having reduced or minimal toxicity.

As defined hereinafter, the term “cholera holotoxin” may be abbreviated as “CT”. As defined hereinafter, a “CT”, a “wild-type CT” and a “mutant CT” are six subunit proteins (i.e., a heterohexamer) comprising five identical (i.e., a homopentamer) cholera toxin B subunits (CT-B) and one (i.e., a monomer) cholera toxin A subunit (CT-A).

As defined hereinafter, a wild-type CT comprises a CT-A subunit polypeptide comprising an amino acid sequence of SEQ ID NO:2. As defined hereinafter, a mutant CT comprises a CT-A subunit polypeptide comprising a genetically modified (i.e., mutated) amino acid sequence of SEQ ID NO:2, wherein the amino acid sequence of SEQ ID NO:2 has been genetically modified at least at amino acid residue Arg-7, Asp-9, Arg-11, Ile-16, Arg-25, Glu-29, Tyr-30, His-44, Val-53, Ser-63, Ser-68, His-70, Val-72, Val-97, Tyr-104, Pro-106, Ser-109, Glu-112 or Arg-192, wherein the mutation at Glu-29 is not an aspartic acid. In a preferred embodiment of the invention, the genetic modification is at amino acid residue 29 of SEQ ID NO:2, wherein the wild-type glutamic acid (E) is mutated to a histidine. (H). Thus, as defined hereinafter, “E29H” refers to a mutant CT polypeptide (i.e., the CT-A subunit of SEQ ID NO:2) having a histidine (H) at amino acid residue 29 of SEQ ID NO:2.

As defined hereinafter, the term “E. coli heat labile toxin” may be abbreviated as “LT.” As defined hereinafter, a “LT”, a “wild-type LT” and a “mutant LT” are six subunit proteins comprising five identical B subunits (LT-B) and one A subunit (LT-A). The LT-A and LT-B polynucleotide and polypeptide sequences are well known in the art, as described in U.S. Pat. No. 6,149,919. As defined hereinafter, the term “Bordetella pertussis toxin” or “pertussis toxin” may be abbreviated as “PT.” As defined hereinafter, a “PT, a wild-type PT” and a “mutant PT” are six subunit proteins comprising five non-identical B subunits (PT-B) and one A subunit (PT-A). The PT-A (also known as subunit S1) and PT-B (also known as subunits S2, S3, S4 and S5) polynucleotide and polypeptide sequences are well known in the art, as described in U.S. Pat. No. 6,350,612 and U.S. Pat. No. 5,785,971.

As defined hereinafter a “mutant PT” or a “mutant LT” comprises a mutation in the A-subunit. Genetic modifications (i.e., mutations) which reduce overall toxicity of PT and LT are well known in the art (International Applications WO 98/42375, WO 93/13202, WO 97/02348 and WO 92/19265).

As defined hereinafter, an “adjuvant,” a “CT adjuvant,” a “PT adjuvant” and a “LT adjuvant” is a composition that serves to enhance the immunogenicity of an antigen. Thus, a mutant CT adjuvant is administered as an adjuvant-antigen conjugate (i.e., covalently associated) such as a mutant CT E29H conjugated with a peptide antigen, a carbohydrate antigen, an oligosaccharide antigen, etc. Similarly, a mutant LT adjuvant or a mutant PT adjuvant is administered as a mutant LT or a mutant PT conjugated with a peptide antigen, a carbohydrate antigen, an oligosaccharide antigen, etc.

The Gram-negative bacterium Vibrio cholerae (V. cholerae) is the causative agent of the gastrointestinal (GI) disease cholera. The diarrhea caused by V. cholerae is due to the secretion of cholera toxin. As defined herein, “reduced toxicity” or “a mutant CT having reduced toxicity” means that the CT mutant exhibits substantially lower toxicity per unit of purified toxin protein compared to the wild-type CT, which allows the mutant CT to be used as an antigen carrier protein having adjuvant activity without causing significant side effects. Similarly, “a mutant LT having reduced toxicity” or “a mutant PT having reduced toxicity” means that the LT or PT mutant exhibits substantially lower toxicity per unit of purified toxin protein compared to the wild-type LT or wild-type PT, respectively, which allows the mutant LT or PT to be used as an antigen carrier protein having adjuvant activity without causing significant side effects.

Thus, in particular embodiments, the invention is directed to a genetically detoxified mutant CT, most preferably the mutant CT E29H. Without eliminating the intrinsic adjuvanting properties of wild-type CT, the CT E29H mutation results in a reduction of the toxicity associated with wild-type CT protein. It is demonstrated in Examples 7-12, that mutant CT E29H functions as a carrier protein for peptide antigens (Examples 7-9), lipooligosaccharide antigens (Example 11) and carbohydrate antigens (Examples 12 and 13), while retaining its intrinsic adjuvant properties. A number of antigens were conjugated to mutant CT E29H using various chemistries. Immunization studies using conjugates of mutant CT E29H and group B Strep antigen (GBSIII); or of mutant CT E29H and the amino-terminal amino acids 1-7 of the 42 amino acid β-amyloid peptide, demonstrate that these conjugates are excellent immunogens in the absence of exogenous adjuvant. For example, in response to both parenteral and intranasal immunization, antibody titers specific for the conjugated antigens were higher than those from the sera of mice immunized with adjuvanted CRM₁₉₇ conjugates after only a single immunization. These results demonstrate that mutant CT E29H functions both as a carrier protein and as an adjuvant which maintains its intrinsic adjuvant properties. In addition, mutant CT E29H conjugates also demonstrate adjuvant activity for non-conjugated, admixed antigens, i.e., as a mutant CT adjuvant (Example 14).

### A. CHOLERA HOLOTOXIN (CT) E. COLI HEAT LABILE TOXIN (LT) AND PERTUSSIS TOXIN (LT) POLYPEPTIDES

In certain embodiments, the invention is directed to compositions and methods of immunization comprising a mutant CT as an antigen carrier protein, wherein the mutant CT has intrinsic adjuvant activity and reduced toxicity compared to a wild-type CT. In certain other embodiments, the invention is directed to compositions and methods of use comprising a mutant CT as an immune adjuvant, wherein the mutant CT has reduced toxicity compared to a wild-type CT. In other embodiments, the invention is directed to a LT or a PT as an adjuvant and an antigen carrier protein, preferably a mutant LT or mutant PT as an adjuvant and an antigen carrier protein.

In particular embodiments, the present invention provides isolated and purified cholera holotoxin polypeptides. Preferably, cholera holotoxin polypeptides of the invention are recombinant polypeptides. As defined hereinafter, a cholera holotoxin (CT) polypeptide is 6 subunit polypeptide comprising 5 identical B subunits (CT-B) and 1 A subunit (CT-A). Thus, a CT polypeptide has a 5:1 stoichiometry of CT-B to CT-A subunits. A wild-type CT of the invention comprises a CT-A subunit comprising an amino acid sequence of SEQ ID NO:2, whereas a mutant CT comprises a CT-A subunit comprising a genetically modified (i.e., mutated) amino acid sequence of SEQ ID NO:2.

In a preferred embodiment, the invention is directed to a mutant CT comprising a CT-A subunit comprising a genetically modified amino acid sequence of SEQ ID NO:2, wherein the amino acid sequence has been genetically modified at least at amino acid residue 29 of SEQ ID NO:2, wherein the modification at residue 29 is not an aspartic acid. In another preferred embodiment of the invention, the genetic modification at amino acid residue 29 of SEQ ID NO:2 is a mutation of the wild-type glutamic acid (E) to a histidine (H). Thus, as defined hereinafter, “E29H” refers to a mutant CT polypeptide (i.e., the CT-A subunit of SEQ ID NO:2) having a istidine (H) at amino acid residue 29 of SEQ ID NO:2.

Alternatively, a genetic modification at amino acid residue 29 of SEQ ID NO:2, may be a mutation (substitution) to an alanine, asparagine, cysteine, phenylalanine, glycine, isoleucine, lysine, leucine, methionine, proline, glutamine, arginine, serine, threonine, valine, tryptophan, or a tyrosine, as long as the CT mutant retains its adjuvant activity and/or reduced toxicity relative to wild-type CT.

In certain other embodiments, the compositions and methods of the present invention comprise a conjugated mutant CT as an adjuvant and an antigen carrier protein, wherein the mutant CT comprises additional mutations including, but not limited to, amino acid residue 29 of SEQ ID NO:2. For Example, U.S. Pat. No. 6,149,919 and International Application WO 93/13202, which are hereby incorporated by reference, describe a series of mutations in the A subunit which serve to reduce the toxicity of the cholera holotoxin. These mutations include making substitutions for the arginine at amino acid 7, the aspartic acid at position 9, the arginine at position 11, the histidine at position 44, the valine at position 53, the arginine at position 54, the serine at position 61, the serine at position 63, the histidine at position 70, the valine at position 97, the tyrosine at position 104, the proline at position 106, the histidine at position 107, the glutamic acid at position 110, the glutamic acid at position 112, the serine at position 114, the tryptophan at position 127, the arginine at position 146 and the arginine at position 192.

International application WO 98/42375, which is hereby incorporated by reference, describes making a substitution for the serine at amino acid 109 in the CT-A subunit, which serves to reduce the toxicity of the cholera holotoxin. International Application WO 97/02348, which is hereby incorporated by reference, describes making a substitution for the serine at amino acid 63 and the arginine at position 192 in the CT-A subunit.

International Application PCT/US02/20978, which is hereby incorporated by reference, describes mutations (substitutions) in the CT-A subunit at isoleucine position 16 (Ile-1 6), valine position 72 (Val-72), double mutations (substitutions) at Ile-16 and Ser-68, and double mutations at Ser-68 and Val-72, all of which serve to reduce toxicity of CT.

International Application PCT/US/21008, which is hereby incorporated by reference, describes both single and double amino acid insertions into the CT-A amino acid sequence which reduce toxicity of CT. For example, an insertion of a single amino acid in the CT-A polypeptide sequence at position 49 (thereby shifting the amino acid residues originally located at positions 49, 50, etc., to positions 50, 51, etc.) is described. Similarly described is an insertion of two amino acids in the CT-A polypeptide sequence at amino acid positions 35 and 36 of the CT-A (thereby shifting the original amino acid residues at positions 35 and 36 to positions 37, 38, etc.). International Application PCT/US/21008 also describes a substitution at amino acid position 30 and an insertion of two amino acids at positions 31 and 32 in the CT-A polypeptide sequence (thereby shifting the original amino acid residues at positions 31 and 32 to positions 33 and 34, etc.).

Therefore, using conventional techniques, mutations and/or insertions at one or more of these additional CT-A positions may be generated, wherein particularly preferred CT-A mutations of SEQ ID NO:2 include amino acid residue Arg-7, Asp-9, Arg-11, Ile-16, Arg-25, Glu-29, Tyr-30, His-44, Val-53, Ser-63, Ser-68, His-70, Val-72, Val-97, Tyr-104, Pro-106, Ser-109, Glu-112 or Arg-192, wherein the mutation at Glu-29 is not an aspartic acid.

The invention, in particular embodiments, is directed to a LT or a PT as an adjuvant and an antigen carrier protein. In preferred embodiments, the LT or PT is a mutant LT or PT having reduced toxicity, such as a mutant PT and a mutant LT described in International Applications WO 98/42375, WO 97/02348, European Patent EP0620850 and U.S. Pat. No. 6,149,919, each incorporated herein by reference in its entirety.

A biological equivalent or variant of a CT polypeptide according to the present invention encompasses a polypeptide that contains substantial homology to a CT polypeptide, as long as the CT-A has a genetic modification at least at amino acid residue 29 of SEQ ID NO:2, wherein the modification at residue 29 is not an aspartic acid. Biological equivalents or variants of CT, LT and PT include CT polypeptides, LT polypeptides or PT polypeptides, which function as an antigen carrier and/or adjuvant.

Functional biological equivalents or variants are naturally occurring amino acid sequence variants of a CT, a LT or a PT polypeptide that maintain the ability to elicit an adjuvant response (i.e., function as an adjuvant) and/or present one or more antigens (i.e., function as an antigen carrier) for immunological response in a subject. Functional variants will typically contain only conservative substitution of one or more amino acids of CT, LT or PT; or substitution, deletion or insertion of non-critical residues in non-critical regions of the CT, LT or PT polypeptide.

Modifications and changes are made in the structure of a polypeptide of the present invention and still obtain a molecule having carrier and/or adjuvant properties. For example, certain amino acids are substituted for other amino acids in a sequence without appreciable loss of adjuvant activity. Because it is the interactive capacity and nature of a polypeptide that defines that polypeptide's biological functional activity, certain amino acid sequence substitutions are made in a polypeptide sequence (or, of course, its underlying DNA coding sequence) and nevertheless obtain a polypeptide with like properties.

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within ±2 is preferred, those that are within ±1 are particularly preferred, and those within ±0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated hereinafter by reference, states that the greatest local average hydrophilicity of a polypeptide, as governed by the hydrophilicity of its adjacent amino acids, correlates with its immunogenicity and antigenicity, i.e. with a biological property of the polypeptide.

As detailed in U.S. Pat. No. 4,554,101, the following hydrophilicity values have been assigned to amino acid residues: arginine (+3.0); lysine (+3.0); aspartate (+3.0 ±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those that are within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (See Table 1, below). The present invention thus contemplates functional or biological equivalents of the polypeptide as set forth above.

TABLE 1
AMINO ACID SUBSTITUTIONS

| | Exemplary Residue |
|---|---|
| Original Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Leu; Tyr |
| Ser | Thr |
| Thr | Ser |
| Trp | Tyr |
| Tyr | Trp; Phe |
| Val | Ile; Leu |

Biological or functional equivalents of a polypeptide are prepared using site-specific mutagenesis. Site-specific mutagenesis is a technique useful in the preparation of second generation polypeptides, or biologically functional equivalent polypeptides or peptides, derived from the sequences thereof, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-directed (site-specific) mutagenesis is well known in the art. As will be appreciated, the technique typically employs a phage vector which exists in both a single stranded and double stranded form. Typically, site-directed mutagenesis in accordance herewith is performed by first obtaining a single-stranded vector which includes within its sequence a DNA sequence which encodes all or a portion of the CT polypeptide sequence selected (i.e., CT-A and CT-B). An oligonucleotide primer bearing the desired mutated sequence is prepared (e.g., synthetically). This primer is then annealed to the singled-stranded vector, and extended by the use of enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells such as E. coli cells and clones are selected which include recombinant vectors bearing the mutation. Commercially available kits come with all the reagents necessary, except the oligonucleotide primers.

A CT polypeptide of the present invention is understood to be any CT polypeptide comprising substantial sequence similarity, structural similarity and/or functional similarity to a CT polypeptide comprising a CT-A having a genetically modified amino acid sequence of SEQ ID NO:2. In addition, a CT polypeptide of the invention is not limited to a particular mutation or a particular source. For example, a CT polypeptide of the invention also comprises one or mutations set forth in U.S Pat. No. 6,149,919, International Application WO 93/13202, International Application WO 98/42375 and International Application WO 97/02348. A LT polypeptide or a PT polypeptide of the present invention is therefore understood to be any LT or PT polypeptide comprising substantial sequence similarity, structural similarity and/or functional similarity to a LT or a PT polypeptide set forth above.

Thus, the invention provides for the general detection and isolation of the polypeptides from a variety of sources, and methods for introducing one or more polypeptide sequence mutations via mutagenesis of the underlying DNA.

### B. CONJUGATED AND NON-CONJUGATED ANTIGENS

In particular embodiments, the invention is directed to compositions and methods of immunization comprising a mutant CT as an antigen carrier protein, wherein the mutant CT antigen has intrinsic adjuvant activity and reduced toxicity compared to a wild-type cholera CT.

In still other embodiments, the invention is directed to compositions and methods of immunization comprising a LT or a PT as an antigen carrier protein, wherein the LT or PT has intrinsic adjuvant activity. In preferred embodiments, the LT or PT is a mutant LT or PT having reduce toxicity relative to wild-type LT or PT.

An antigen is typically defined on the basis of immunogenicity. Immunogenicity is defined as the ability to induce a humoral and/or cell-mediated immune response. Thus, the terms antigen or immunogen, as defined hereinafter, are molecules possessing the ability to induce a humoral and/or cell-mediated immune response.

Antigens contemplated for use in the present invention are such molecules that can induce a specific immune response. In certain preferred embodiments, an antigen is a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate, a polysaccharide-protein conjugate, or any combination thereof.

Where a mutant CT, a mutant LT or a mutant PT and one or more antigens are conjugated (i.e., covalently associated), conjugation may be any chemical method, process or genetic technique commonly used in the art. For example, a mutant CT polypeptide and one or more antigens selected from a polypeptide, polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate, a polysaccharide-protein conjugate, or any combination thereof, may be conjugated by techniques, including, but not limited to: (1) direct coupling via protein functional groups (e.g., thiol-thiol linkage, amine-carboxyl linkage, amine-aldehyde linkage; enzyme direct coupling); (2) homobifunctional coupling of amines (e.g., using bis-aldehydes); (3) homobifunctional coupling of thiols (e.g., using bis-maleimides); (4) homobifunctional coupling via photoactivated reagents (5) heterobifunctional coupling of amines to thiols (e.g., using maleimides); (6) heterobifunctional coupling via photoactivated reagents (e.g., the β-carbonyldiazo family); (7) introducing amine-reactive groups into a poly- or oligosaccharide via cyanogen bromide activation or carboxymethylation; (8) introducing thiol-reactive groups into a poly- or oligosaccharide via a heterobifunctional compound such as maleimido-hydrazide; (9) protein-lipid conjugation via introducing a hydrophobic group into the protein and (10) protein-lipid conjugation via incorporating a reactive group into the lipid. Also, contemplated are heterobifunctional “non-covalent coupling” techniques such the Biotin-Avidin interaction. For a comprehensive review of conjugation techniques, see Aslam and Dent (1998), incorporated hereinafter by reference in its entirety.

### C. POLYNUCLEOTIDES ENCODING CHOLERA HOLOTOXIN (CT), HEAT LABILE TOXIN (LT) AND PERTUSSIS TOXIN (PT)

Isolated and purified CT, LT and PT polynucleotides of the present invention are contemplated for use in the production of CT, LT and PT polypeptides. More specifically, in certain embodiments, the polynucleotides encode CT polypeptides, particularly CT-B subunits and wild-type CT-A subunits or genetically modified CT-A subunits.

In particular embodiments, a polynucleotide of the present invention is a DNA molecule, wherein the DNA may be genomic DNA, chromosomal DNA, plasmid DNA or cDNA. In a preferred embodiment, a polynucleotide of the present invention is a recombinant polynucleotide, which encodes a mutant CT polypeptide (i.e., a mutant CT-A), wherein the CT-A comprises a genetically modified amino acid sequence of SEQ ID NO:2.

As used hereinafter, the term “polynucleotide” means a sequence of nucleotides connected by phosphodiester linkages. Polynucleotides are presented hereinafter in the 5′ to the 3′ direction. A polynucleotide of the present invention comprises from about 10 to about several hundred thousand base pairs. Preferably, a polynucleotide comprises from about 10 to about 3,000 base pairs. Preferred lengths of particular polynucleotide are set forth hereinafter.

A polynucleotide of the present invention can be a deoxyribonucleic acid (DNA) molecule, a ribonucleic acid (RNA) molecule, or analogs of the DNA or RNA generated using nucleotide analogs. The nucleic acid molecule can be single-stranded or double-stranded, but preferably is double-stranded DNA. Where a polynucleotide is a DNA molecule, that molecule can be a gene, a cDNA molecule or a genomic DNA molecule. Nucleotide bases are indicated hereinafter by a single letter code: adenine (A), guanine (G), thymine (T), cytosine (C), inosine (I) and uracil (U).

“Isolated” means altered “by the hand of man” from the natural state. If an “isolated” composition or substance occurs in nature, it has been changed or removed from its original environment, or both. For example, a polynucleotide or a polypeptide naturally present in a living animal is not “isolated,” but the same polynucleotide or polypeptide separated from the coexisting materials of its natural state is “isolated,” as the term is employed hereinafter.

Preferably, an “isolated” polynucleotide is free of sequences which naturally flank the nucleic acid (i.e., sequences located at the 5′ and 3′ ends of the nucleic acid) in the genomic DNA of the organism from which the nucleic acid is derived.

Polynucleotides of the present invention are obtained, using standard cloning and screening techniques, from a cDNA library derived from mRNA. Polynucleotides of the invention are also obtained from natural sources such as genomic DNA libraries (e.g., a Vibrio cholera library) or synthesized using well known and commercially available techniques.

Orthologues and allelic variants of the CT, LT or PT polynucleotides can readily be identified using methods well known in the art. Allelic variants and orthologues of the CT polynucleotides will comprise a nucleotide sequence that is typically at least about 70-75%, more typically at least about 80-85%, and most typically at least about 90-95% or more homologous to the CT nucleotide sequence shown in SEQ ID NO:1, or a fragment of this nucleotide sequence. Such nucleic acid molecules can readily be identified as being able to hybridize, preferably under stringent conditions, to the CT nucleotide sequence shown in SEQ ID NO:1, or a fragment of this nucleotide sequence.

When the CT, LT or PT polynucleotides of the invention are used for the recombinant production of CT, LT or PT polypeptides of the present invention, the polynucleotide includes the coding sequence for the mature polypeptide, by itself, or the coding sequence for the mature polypeptide in reading frame with other coding sequences, such as those encoding a leader or secretory sequence, a pre-, a pro- a prepro-protein sequence, or other fusion peptide portions. For example, a marker sequence which facilitates purification of the fused polypeptide can be linked to the coding sequence (see Gentz et al., 1989, incorporated by reference hereinafter in its entirety). Thus, contemplated in the present invention is the preparation of polynucleotides encoding fusion polypeptides permitting His-tag purification of expression products. The polynucleotide may also contain non-coding 5′ and 3′ sequences, such as transcribed, non-translated sequences, splicing and polyadenylation signals.

In certain embodiments, it is advantageous to use oligonucleotide primers. These primers may be generated in any manner, including chemical synthesis, DNA replication, reverse transcription, or a combination thereof. The sequence of such primers is designed using a polynucleotide of the present invention for use in detecting, amplifying or mutating a defined segment of a polynucleotide from prokaryotic cells using polymerase chain reaction (PCR) technology.

Polynucleotides which are identical or sufficiently identical to a CT, LT or PT nucleotide sequence or a fragment thereof, may be used as hybridization probes for DNA and genomic DNA or as primers for a nucleic acid amplification (PCR) reaction, to isolate full-length cDNAs and genomic clones encoding polypeptides of the present invention and to isolate cDNA and genomic clones of other genes (including genes encoding homologs and orthologs from species other than Vibrio Cholera) that have a high sequence similarity to the CT, LT or PT polynucleotide sequence or a fragment thereof. Typically these nucleotide sequences are from at least about 70% identical to at least about 95% identical to that of the reference polynucleotide sequence. The probes or primers will generally comprise at least 15 nucleotides, preferably, at least 30 nucleotides and may have at least 50 nucleotides. Particularly preferred probes will have between 30 and 50 nucleotides.

There are several methods available and well known to those skilled in the art to obtain full-length cDNAs, or extend short cDNAs, for example those based on the method of Rapid Amplification of cDNA ends (RACE) (see, Frohman et al., 1988). Recent modifications of the technique, exemplified by the Marathon™ technology (Clontech Laboratories Inc.) for example, have significantly simplified the search for longer cDNAs. In the Marathon™ technology, cDNAs have been prepared from mRNA extracted from a chosen tissue and an “adaptor” sequence ligated onto each end. Nucleic acid amplification (PCR) is then carried out to amplify the “missing” 5′ end of the cDNA using a combination of gene specific and adaptor specific oligonucleotide primers. The PCR reaction is then repeated using “nested” primers, that is, primers designed to anneal within the amplified product (typically an adaptor specific primer that anneals further 3′ in the adaptor sequence and a gene specific primer that anneals further 5′ in the known gene sequence). The products of this reaction are then analyzed by DNA sequencing and a full-length cDNA constructed either by joining the product directly to the existing cDNA to give a complete sequence, or carrying out a separate full-length PCR using the new sequence information for the design of the 5′ primer.

In another embodiment, a polynucleotide probe molecule of the invention can be used for its ability to selectively form duplex molecules with complementary stretches of the gene. Depending on the application envisioned, one will desire to employ varying conditions of hybridization to achieve varying degree of selectivity of the probe toward the target sequence (see Table 2 below). For applications requiring a high degree of selectivity, one will typically desire to employ relatively stringent conditions to form the hybrids. For some applications, for example, where one desires to prepare mutants employing a mutant primer strand hybridized to an underlying template or where one seeks to isolate a homologous polypeptide coding sequence from other cells, functional equivalents, or the like, less stringent hybridization conditions are typically needed to allow formation of the heteroduplex (see Table 2). Cross-hybridizing species can thereby be readily identified as positively hybridizing signals with respect to control hybridizations. Thus, hybridization conditions are readily manipulated, and thus will generally be a method of choice depending on the desired results.

For some applications, for example, where one desires to prepare mutants employing a mutant primer strand hybridized to an underlying template or where one seeks to isolate a homologous polypeptide coding sequence from other cells, functional equivalents, or the like, less stringent hybridization conditions are typically needed to allow formation of the heteroduplex. Cross-hybridizing species -are thereby readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions are readily manipulated, and thus will generally be a method of choice depending on the desired results.

The present invention also includes polynucleotides capable of hybridizing under reduced stringency conditions, more preferably stringent conditions, and most preferably highly stringent conditions, to polynucleotides described hereinafter. Examples of stringency conditions are shown in Table 2 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 2
Hybridization Stringency Conditions

| | | | Hybridization | Wash |
|---|---|---|---|---|
| Stringency | Polynucleotide | Hybrid Length | Temperature and | Temperature |
| Condition | Hybrid | (bp)^{I} | Buffer^{H} | and Buffer^{H} |
| A | DNA:DNA | >50 | 65° C.; 1 × SSC -or- | 65° C.; |
| | | | 42° C.; 1 × SSC, 50% | 0.3 × SSC |
| | | | formamide | |
| B | DNA:DNA | <50 | T_{B}; 1 × SSC | T_{B}; 1 × SSC |
| C | DNA:RNA | >50 | 67° C.; 1 × SSC -or- | 67° C.; |
| | | | 45° C.; 1 × SSC, 50% | 0.3 × SSC |
| | | | formamide | |
| D | DNA:RNA | <50 | T_{D}; 1 × SSC | T_{D}; 1 × SSC |
| E | RNA:RNA | >50 | 70° C.; 1 × SSC -or- | 70° C.; |
| | | | 50° C.; 1 × SSC, 50% | 0.3 × SSC |
| | | | formamide | |
| F | RNA:RNA | <50 | T_{F}; 1 × SSC | T_{F}; 1 × SSC |
| G | DNA:DNA | >50 | 65° C.; 4 × SSC -or- | 65° C.; 1 × SSC |
| | | | 42° C.; 4 × SSC, 50% | |
| | | | formamide | |
| H | DNA:DNA | <50 | T_{H}; 4 × SSC | T_{H}; 4 × SSC |
| I | DNA:RNA | >50 | 67° C.; 4 × SSC -or- | 67° C.; 1 × SSC |
| | | | 45° C.; 4 × SSC, 50% | |
| | | | formamide | |
| J | DNA:RNA | <50 | T_{J}; 4 × SSC | T_{J}; 4 × SSC |
| K | RNA:RNA | >50 | 70° C.; 4 × SSC -or- | 67° C.; 1 × SSC |
| | | | 50° C.; 4 × SSC, 50% | |
| | | | formamide | |
| L | RNA:RNA | <50 | T_{L}; 2 × SSC | T_{L}; 2 × SSC |
| M | DNA:DNA | >50 | 50° C.; 4 × SSC -or- | 50° C.; 2 × SSC |
| | | | 40° C.; 6 × SSC, 50% | |
| | | | formamide | |
| N | DNA:DNA | <50 | T_{N}; 6 × SSC | T_{N}; 6 × SSC |
| O | DNA:RNA | >50 | 55° C.; 4 × SSC -or- | 55° C.; 2 × SSC |
| | | | 42° C.; 6 × SSC, 50% | |
| | | | formamide | |
| P | DNA:RNA | <50 | T_{P}; 6 × SSC | T_{P}; 6 × SSC |
| Q | RNA:RNA | >50 | 60° C.; 4 × SSC -or- | 60° C.; 2 × SSC |
| | | | 45° C.; 6 × SSC, 50% | |
| | | | formamide | |
| R | RNA:RNA | <50 | T_{R}; 4 × SSC | T_{R}; 4 × SSC |
| (bp)^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length is determined by aligning the sequences of the polynucleotides and identifying the region or regions of optimal sequence complementarity. | | | | |
| Buffer^{H}: SSPE (1 × SSPE is 0.15 M NaCl, 10 mM NaH₂PO₄, and 1.25 mM EDTA, pH 7.4) can be substituted for SSC (1 × SSC is 0.15 M NaCl and 15 mM sodium citrate) in the hybridization and wash buffers; washes are performed for 15 minutes after hybridization is complete. | | | | |
| T_{B} through T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T | | | | |
| # bases) + 4(# of G + C bases). For hybrids between 18 and 49 base pairs in length, Tₘ(° C.) = 81.5 + 16.6(log₁₀[Na⁺]) + 0.41 (% G + C) − (600/N), where N is the number of bases in the hybrid, and [Na⁺] is the concentration of sodium ions in the hybridization buffer ([Na⁺] for 1 × SSC = 0.165 M). | | | | |

Additional examples of stringency conditions for polynucleotide hybridization are provided in Sambrook et al., 1989, Molecular Cloning: A Laboratory Manual, Cold Spring Harbor Laboratory Press, Cold Spring Harbor, N.Y., chapters 9 and 11, and Ausubel et al., 1995, Current Protocols in Molecular Biology, eds., John Wiley & Sons, Inc., sections 2.10 and 6.3-6.4, incorporated hereinafter by reference.

### D. IMMUNOGENIC AND PHARMACEUTICAL COMPOSITIONS

CT, LT or PT polypeptide-antigen conjugates of the present invention are incorporated into pharmaceutical and immunogenic compositions suitable for administration to a subject, e.g., a human. Such compositions typically comprise the “active” composition and a pharmaceutically acceptable carrier. As used hereinafter the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, such media can be used in the compositions of the invention. Supplementary active compounds can also be incorporated into the compositions.

A pharmaceutical or immunogenic composition of the invention is formulated to be compatible with its intended route of administration. Examples of routes of administration include parenteral. (e.g., intravenous, intradermal, subcutaneous, intramuscular, intraperitoneal), mucosal (e.g., oral, rectal, intranasal, buccal, vaginal, respiratory) and transdermal (topical). Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH is adjusted with acids or bases, such as hydrochloric acid or sodium hydroxide. The parenteral preparation is enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier is a solvent or dispersion medium containing, for example, water, ethanol, polyol (e.g., glycerol, propylene glycol, and liquid polyetheylene glycol, and the like), and suitable mixtures thereof. The proper fluidity is maintained, for example, by the use of a coating such as lecithin, by the maintenance of the required particle size in the case of dispersion and by the use of surfactants. Prevention of the action of microorganisms is achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, 'sodium chloride in the composition. Prolonged absorption of the injectable compositions can be brought about by including in the composition an agent which delays absorption, for example, aluminum monostearate and gelatin.

Sterile injectable solutions are prepared by incorporating the active compound (e.g., a mutant CT-antigen conjugate) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They are enclosed in gelatin capsules or compressed into tablets. For the purpose of oral therapeutic administration, the active compound is incorporated with excipients and used in the form of tablets, troches, or capsules. Oral compositions are also prepared using a fluid carrier for use as a mouthwash, wherein the compound in the fluid carrier is applied orally and swished and expectorated or swallowed. Pharmaceutically compatible binding agents, and/or adjuvant materials are included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Systemic administration is by mucosal or transdermal means. For mucosal or transdermal administration, penetrants appropriate to the barrier to be permeated are used in the formulation. Such penetrants are generally known in the art, and include, for example, for transmucosal administration, detergents, bile salts, and fusidic acid derivatives. Mucosal administration is accomplished through the use of nasal sprays or suppositories. For transdermal administration, the active compounds are formulated into ointments, salves, gels, or creams as generally known in the art.

The compounds are also prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems.

Biodegradable, biocompatible polymers are used, such as ethylene vinyl acetate, polyanhydrides, polyglycolic acid, collagen, polyorthoesters, and polylactic acid. Methods for preparation of such formulations will be apparent to those skilled in the art. The materials are obtained commercially from Alza Corporation and Nova Pharmaceuticals, Inc. Liposomal suspensions are also used as pharmaceutically acceptable carriers. These are prepared according to methods known to those skilled in the art, for example, as described in U.S. Pat. No. 4,522,811 which is incorporated hereinafter by reference.

It is especially advantageous to formulate oral or parenteral compositions in dosage unit form for ease of administration and uniformity of dosage. “Dosage unit form” as used hereinafter refers to physically discrete units suited as unitary dosages for the subject to be treated; each unit containing a predetermined quantity of active compound is calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Combination immunogenic compositions are provided by including two or more of the polypeptides of the invention (e.g., one or more mutant CT-conjugates, with or without one or more unconjugated antigens). In particular, combination immunogenic compositions are provided by combining one or more of the CT-conjugates of the invention with one or more polypeptide, polypeptide fragment, carbohydrate, oligosaccharide, lipid, lipooligosaccharide, polysaccharide, oligosaccharide-protein conjugate, polysaccharide-protein conjugate, peptide-protein conjugate, oligosaccharide-peptide conjugate, polysaccharide-peptide conjugate,. protein-protein conjugate, lipooligosaccharide-protein conjugate or polysaccharide-protein conjugate.

A pharmaceutically acceptable vehicle is understood to designate a compound or a combination of compounds entering into a pharmaceutical or immunogenic composition which does not cause side effects and which makes it possible, for example, to facilitate the administration of the active compound, to increase its life and/or its efficacy in the body, to increase its solubility in solution or alternatively to enhance its preservation. These pharmaceutically acceptable vehicles are well known and will be adapted by persons skilled in the art according to the nature and the mode of administration of the active compound chosen.

As defined previously, an “adjuvant” is a substance that serves to enhance the immunogenicity of an antigen. Thus, adjuvants are often given to boost the immune response and are well known to the skilled artisan. Examples of adjuvants contemplated in the present invention include, but are not limited to, aluminum salts (alum) such as aluminum phosphate and aluminum hydroxide, Mycobacterium tuberculosis, Bordetella pertussis, bacterial lipopolysaccharides, aminoalkyl glucosamine phosphate compounds (AGP), or derivatives or analogs thereof, which are available from Corixa (Hamilton, Mont.), and which are described in U.S. Pat. No. 6,113,918; one such AGP is 2-[(R)-3-Tetradecanoyloxytetradecanoylamino]ethyl 2-Deoxy-4-O-phosphono-3-O-[(R)-3-tetradecanoyoxytetradecanoyl]-2-[(R)-3-tetradecanoyoxytetradecanoylamino]-b-D-glucopyranoside, which is also known as 529 (formerly known as RC529), which is formulated as an aqueous form or as a stable emulsion, MPL™ (3-O-deacylated monophosphoryl lipid A) (Corixa) described in U.S. Pat. No. 4,912,094, synthetic polynucleotides such as oligonucleotides containing a CpG motif (U.S. Pat. No. 6,207,646), polypeptides, saponins such as Quil A or STIMULON™ QS-21 (Antigenics, Framingham, Mass.), described in U.S. Pat. No. 5,057,540, a pertussis toxin (PT), or an E. coli heat-labile toxin (LT), particularly LT-K63, LT-R72, CT-S109, PT-K9/G129; see, e.g., International Patent Publication Nos. WO 93/13302 and WO 92/19265, cholera toxin (either in a wild-type or mutant form, e.g., wherein the glutamic acid at amino acid position 29 is replaced by another amino acid, preferably a histidine, in accordance with published International Patent Application number WO 00/18434). Various cytokines and lymphokines are. suitable for use as adjuvants. One such adjuvant is granulocyte-macrophage colony stimulating factor (GM-CSF), which has a nucleotide sequence as described in U.S. Pat. No. 5,078,996. A plasmid containing GM-CSF cDNA has been transformed into E. coli and has been deposited with the American Type Culture Collection (ATCC), 1081 University Boulevard, Manassas, Va. 20110-2209, under Accession Number 39900. The cytokine Interleukin-12 (IL-12) is another adjuvant which is described in U.S. Pat. No. 5,723,127. Other cytokines or lymphokines have been shown to have immune modulating activity, including, but not limited to, the interleukins 1-α, 1-β, 2, 4, 5, 6, 7, 8, 10, 13, 14, 15, 16, 17 and 18, the interferons-α, β and γ, granulocyte colony stimulating factor, and the tumor necrosis factors α and β, and are suitable for use as adjuvants.

All patents and publications cited herein are hereby incorporated by reference.

### G. EXAMPLES

The following examples are carried out using standard techniques, which are well known and routine to those of skill in the art, except where otherwise described in detail. The following examples are presented for illustrative purpose, and should not be construed in any way limiting the scope of this invention.

### Example 1

### Bacterial Strains, Plasmids and Growth Conditions

E. coil TG1 (Amersham-Pharmacia Biotech, Piscataway, N.J.), and TX1, a nalidixic acid-resistant derivative of TG1, carrying FTc,lacI^{q} from XL1 blue (Stratagene, LaJolla, Calif.; (Jobling and Holmes, 1992)) and CJ236(FTc, lacI^{q}) (Bio-Rad, Hercules, Calif.) were used as hosts for cloning recombinant plasmids and expression of mutated proteins. Plasmid-containing strains were maintained on LB agar plates with antibiotics as required (ampicillin, 50 μg/ml; kanamycin 25 μg/ml; tetracycline 10 μg/ml). A complete CT operon from V. cholerae 0395 was subcloned into the phagemid vector pSKII⁻, under the control of the lac promoter, to create the IPTG inducible plasmid designated pMGJ67 (Jobling and Holmes, 1991).

### Example 2

### Mutagensis of ctxA Gene

The method of Kunkel (Kunkel, 1985) was used to select for oligonucleotide-derived mutants created in plasmid pMGJ67. The oligonucleotides used to generate five mutant CT-A subunits are described in Table 3.

TABLE 3

Sequence of Oligonucleotides Introduced
into ctxA

| | |
|---|---|
| Substitution | Oligonucleotide Sequence^{a} |

| | | |
|---|---|---|
| R7K | AAGTTATATAAGGCAGATTC | (SEQ ID NO:3) |
| R11K | CAGATTCTAAACCTCCTG | (SEQ ID NO:4) |
| E29H | GACAGAGTNAGTACTTTGACCG | (SEQ ID NO:5) |
| E110D | CAGATGAKCAAGAKGTTTCTGC | (SEQ ID NO:6) |
| E112D | CAGATGAKCAAGAKGTTTCTGC | (SEQ ID NO:7) |

^{a}Altered bases are underlined; N = any base; K = T or G.

Briefly, each single-stranded oligonucleotide was phosphorylated and used to direct second strand synthesis on a uracil-containing single-stranded DNA template rescued from the E. coli dut una strain CJ236(F′Tc, pMGJ67). Following ligation and transformation of ung⁺ strain TX1, single-stranded DNA was rescued from Amp^{R} transformants and sequenced by the dideoxy chain termination method (Sanger, 1977).

### Example 3

### Construction of the Plasmid Encoding CT E29H

The plasmid encoding CT E29H is designated pIIB29H. The plasmid contains the polycistron of V. cholerae genes ctxA and ctxB which encode CT. The ctxA gene in this plasmid was mutagenized as described above to encode a histidine at amino acid position 29 of CT-A. The wild-type polycistron was also altered by removing the native ToxR inducible promoter and replacing it with a lactose inducible promoter. Furthermore, the regions encoding the cbcA and ctxB signal sequences were replaced with the signal sequence-encoding region of E. coli LT (LTIIb-B leader) in order to promote secretion of CT E29H. The plasmid pIIB29H was then modified in an attempt to increase the expression of CT-E29H. The resulting plasmid, designated pPX2492, contained synthetic Shine-Dalgarno sequences upstream of each of ctxA and ctxB. The two genes are genetically separated in pPX2492, unlike in V. cholerae, where the genes overlap. The two genes also have the LTIIb-B leader sequence upstream of each.

### Example 4

### Expression of Mutant CTXA Alleles

Production of each variant holotoxin was tested in 5 ml cultures of Terrific Broth medium (Tartof and Hobbs, 1987) in 125 ml Erlenmeyer flasks at 37° C. with shaking (200 rpm). Logarithmic phase cells (A₆₀₀=0.8-1.0) were induced by the addition of IPTG to 0.4 mM, followed by growth overnight. Polymyxin B was added to 1 mg/ml, followed by incubation for 10 minutes at 37° C. Cells were removed by centrifugation, and the supernatants were assayed to determine the concentrations of holotoxin and B pentamer as described below.

Specifically, the production of CT E29H in E. coli involves the co-expression of the genes rpoH from E. coli and dsbA from V. cholerae. These gene products participate in the conformational maturation of both the CT-A and CT-B subunits of CT holotoxin.

### Example 5

### Aβ 1-7 Peptide Synthesis and Purification

Aβ 1-7 peptide was synthesized on the Rainin Symphony peptide synthesizer using the fluoromethoxy carbonyl (Fmoc) blocking group O-Benzotriazol-1-yl-N,N,N′,N′-tetramethyluronium hexafluorophosphate (HBTU), double couple chemistry with a four-fold reagent excess of amino acids and equimolar excess of HBTU (i.e., 1:1 amino acid:HBTU).

Crude peptides were cleaved from the Wang resin via 95% trifluoroacetic acid (TFA) plus scavengers for 2.5 hours at room temperature. Peptides were purified via reverse phase, semi-preparative HPLC using a Vydac C-18 column (catalogue No. 218TP510), using a 30 minute gradient (5-60% mobile phase of 0.1% TFA/CH3CN ) with a flow rate of 7 mLs/minute.

The Aβ 1-7 peptide has the following amino acid sequence: DAEFRHD (SEQ ID NO:8)

### Example 6

### Aβ 1-7 Peptide/CT E29H Conjugation

CT E29H was bromoacetylated, and the activated CT E29H protein was conjugated to the trifluoroacetylated blocked derivative of the Aβ 1-7 peptide. Mass spectrometry verified activation of CT E29H, and amino acid analysis confirmed conjugation of the peptide to the toxoid. Western blot analysis using MAb 3D6, specific for the N-terminus of the Aβ 1-7 peptide, suggested that the peptide was conjugated to both α/γ and the β subunits of the toxoid molecule.

CT E29H (5 ml at 2 mg/ml) was mixed with N-succinimidyl bromoacetate (Sigma B-8271) at a ratio of 0.9:1 (w/w) in PBS/0.1 M bicarbonate buffer for one hour at room temperature. Excess activator was removed with a P6-DG desalting column. Bromoacetylated CT E29H was analyzed by mass spectrometry, then mixed with Aβ 1-7 peptide at a ratio of 1:1 (w/w) at a final protein and peptide concentration of 1.2 mg/mL and a pH of 9.0. The reaction was mixed overnight at 4° C., then dialyzed against 10 mM NaPO₄, 150 mM NaCl, pH 7.1. The sample was analyzed by amino acid analysis, and also run on SDS-PAGE and Western blot.

Mass spectra analysis of CT E29H before and after bromoacetylation showed that the major peak is that of the β-chain of the toxoid molecule (expected MW is 11,644 Da). This was expected since there are five β-chains in CT E29H per α/γ chain. Minor peaks in the pre-activation spectrum may include: the double charge of the β-chain (expected MW=5,822 Da), a β-chain dimmer (expected MW=23, 288), an α/γ chain species (expected MW=27,210 Da), a β-chain trimer (expected MW=34,932 Da), an α/β/γ aggregate (expected MW=38, 854 Da) and an α/γ chain dimmer (expected MW=54,420 Da). Minor peaks indicative of these possibilities were present. By subtracting each peak from the non-activated sample from its counterpart in the activated sample, an estimate was made of the number of lysines bromoacetylated for each species.

After the bromoacetylated material was incubated overnight in the presence of peptide, the covalent linkage of peptide and CT E29H was verified by two methods: amino acid analysis, and Western blot analysis. Amino acid analysis reported that 13.26 moles of carboxymethylcysteine were recovered per mole of CT E29H. The Western blot analysis verified that only the peptide/CT E29H conjugate reacted with a monoclonal antibody specific for the N-terminus of Aβ 1-7 peptide, while neither the CT E29H holotoxin or the activated CT E29H showed reactivity. The Western blot analysis also indicated that multiple fragments of CT E29H were modified, since there were several species that the mAb (3D6) recognized (data not shown). The molecular weight of these fragments was determined to be 10, 33, 40, and 50 kDa. Without an antibody specific for the different chains of CT E29H, it is unclear which chain these different species correspond to.

### Example 7

### Parenteral Immunogenicity Studies

Several studies were conducted in mice to evaluate the immunogenicity of the Aβ 1-7 peptide/CT E29H conjugate. As a prototypic peptide conjugate, the first seven N-terminal amino acids of the amyloid beta peptide were conjugated as described in Example 6. In the first of several studies, groups of five Swiss Webster female mice were immunized with 5 ug (total protein) of the indicated conjugates, 30 ug of Aβ 1-42 peptide, 10 ug GMCSF, 5 ug CT E29H, or 25 ug 529SE as indicated. Mice were immunized subcutaneously on weeks 0 and 3. Antigen(s) was mixed with or without the indicated adjuvant, and phosphate buffered saline or saline, such that the final immunization volume was 0.2 ml. The immunization volume was divided equally into each of two sites at the base of the tail in the rump area. Individual sera were collected and measured for peptide specific IgG antibody titers prior to immunization, the day prior to the second immunization, and two weeks thereafter. As for all ELISA analysis, endpoint titers were determined using an optical density cut off value of 0.1.

An antigen-specific ELISA was used to measure endpoint titers of sera. Briefly, dilutions of murine sera were added to 96 well ELISA plates coated with appropriate antigen (Aβ 1-42) and blocked. Antigen-specific antibody was then evaluated using biotinylated polyclonal antibody specific for IgG or subclasses thereof. Assays were developed and read at OD of 405 nm after development using a strepavidin HRP conjugate. Titers were determined using Softmax Pro software.

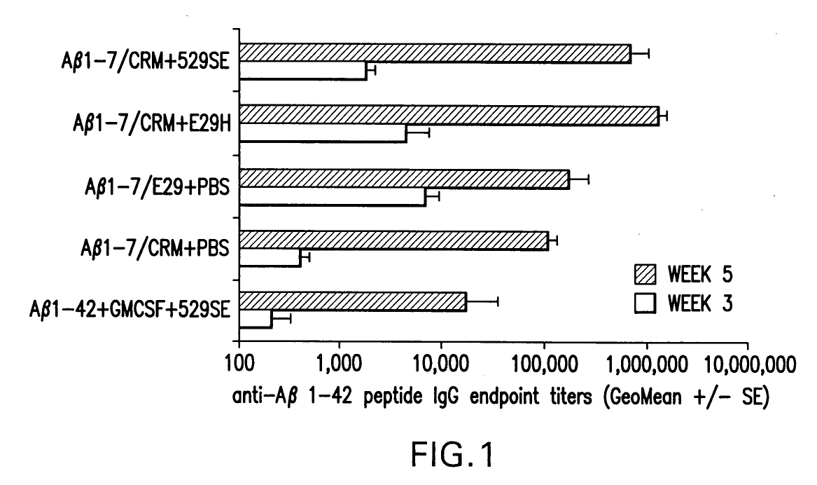

An exemplary carrier protein having adjuvant properties is diphtheria toxin CRM₁₉₇ (a non-toxic form of diphtheria toxin, see U.S. Pat. No. 5,614,382). It was also desirable to determine if a conjugate of CT E29H and Aβ 1-7 peptide demonstrated enhanced antibody responses when compared with peptide conjugates of CRM₁₉₇, with or without addition of supplemental adjuvant. The results demonstrate that CT E29H is an effective carrier for the 7 amino acid Aβ 1-7 peptide (FIG. 1).

The data are summarized as follows: After a single injection, Aβ 1-7 peptide/CT E29H conjugate induced peptide-specific IgG titers that were at least 8-fold higher than those measured from mice immunized with non-adjuvanted Aβ 1-7 peptide/CRM₁₉₇ conjugated peptide. Peptide-specific IgG titers measured from mice immunized with the Aβ 1-7 peptide/CT E29H conjugate were similar to those measured from sera of mice immunized with Aβ/1-7 peptide/CRM₁₉₇ conjugated material separately adjuvanted with either 529SE or CT E29H. CT E29H is a potent parenteral adjuvant for CRM₁₉₇ conjugates. One week after a second injection, mice immunized with Aβ 1-7 peptide/CT E29H conjugates had higher titers than mice immunized with Aβ 1-7 peptide/CRM₁₉₇ conjugate. The adjuvant effect was not as evident as in response to the initial priming immunization. At all time points evaluated, in this and subsequent studies, all Aβ 1-7 peptide conjugates induced higher peptide-specific IgG titers than did Aβ 1-42 formulated with 529SE and GM-CSF.

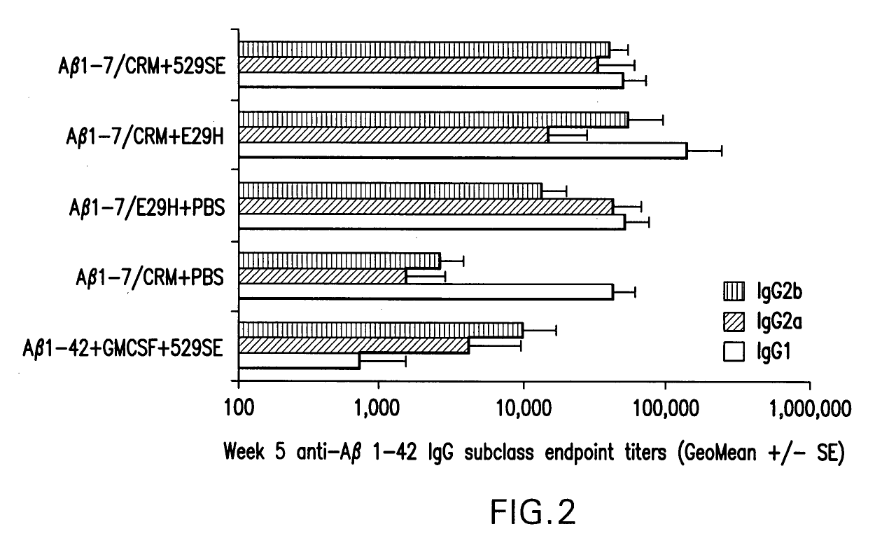

An analysis of peptide-specific IgG subclass titer distribution demonstrates that conjugation of the first 7 amino acids of the Aβ 1-7 peptide to CT E29H results in higher titers, and a distribution profile similar to that seen in mice immunized with adjuvanted (either CT E29H or 529SE) Aβ 1-7 peptide/CRM₁₉₇ conjugate. When compared to the titers of mice immunized with non-adjuvanted (PBS) Aβ 1-7 peptide/CRM₁₉₇ conjugate, the titers of mice immunized with Aβ 1-7 peptide/CT E29H conjugate had higher IgG2a and IgG2b peptide-specific titers (FIG. 2).

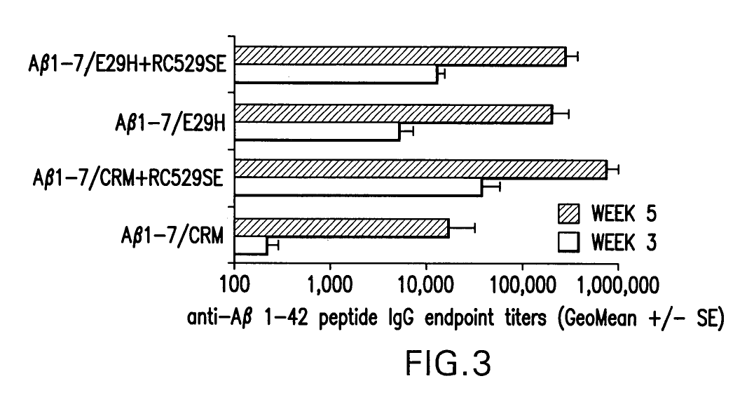

In a separate study, similar results were obtained. Mice immunized with an Aβ 1-7 peptide/CT E29H conjugate demonstrated peptide-specific primary response IgG titers that were approximately one log (10-fold) higher than those determined from mice immunized with a non-adjuvanted Aβ 1-7 peptide/CRM₁₉₇ conjugate (FIG. 3). In this study, 10 Swiss Webster female mice were immunized as described above. In this and in other studies, significant increases were not observed in peptide-specific IgG or subclass titers by the addition of 529SE adjuvant to the Aβ 1-7 peptide/CT E29H conjugate. In contrast, the co-formulation of the Aβ 1-7 peptide/CRM₁₉₇ conjugate with 529SE resulted in significantly enhanced peptide-specific IgG titers (FIG. 3).

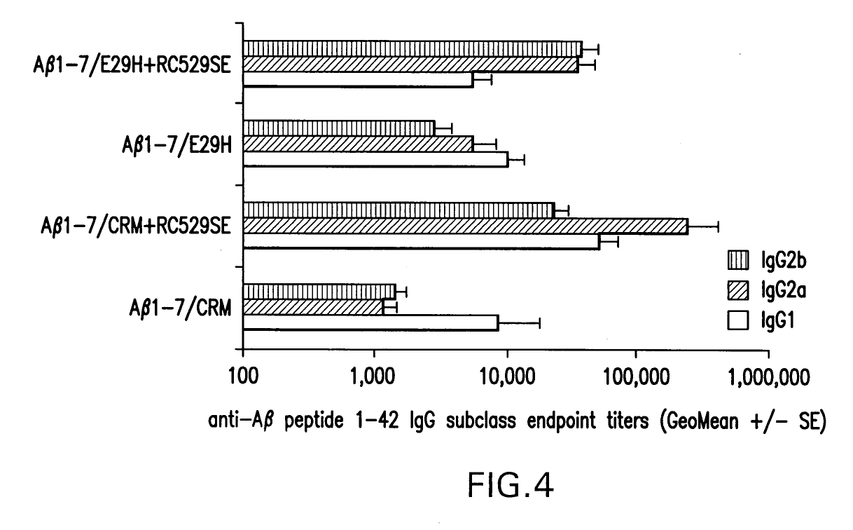

As in the previous study, peptide-specific IgG1 titers were similar for groups of mice immunized with either non-adjuvanted CRM₁₉₇ conjugate, or with the CT E29H conjugate. Peptide-specific IgG2a and IgG2b titers measured from week 5 sera were elevated in the mice immunized with the Aβ 1-7 peptide/CT E29H conjugate with 529SE as compared to those in mice immunized with Aβ 1-7 peptide/CT E29H conjugate without 529SE (FIG. 4).

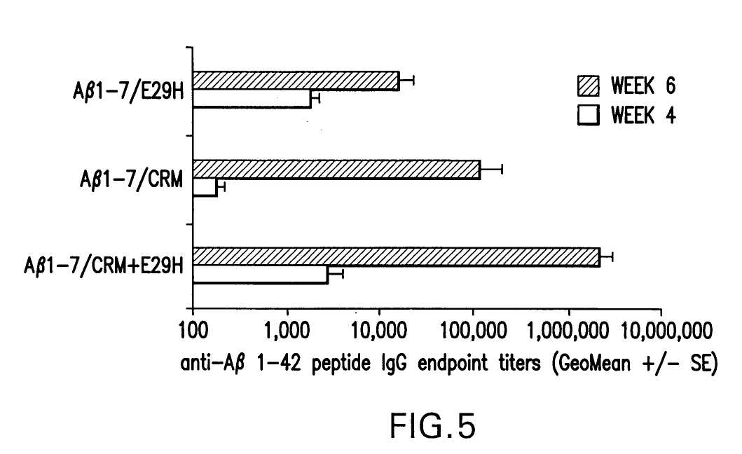

In a study using Balb/c mice, similar results were obtained. Balb/c female mice were immunized with non-adjuvanted CT E29H or CRM₁₉₇-peptide conjugate, or with the peptide-CRM₁₉₇ conjugate adjuvanted with 1 ug of non-conjugated CT E29H (FIG. 5). As in studies with Swiss Webster mice, Balb/c mice responded with higher primary response titers upon immunization with the Aβ 1-7 peptide/CT E29H conjugate than to immunization with the Aβ 1-7 peptide/CRM₁₉₇ conjugate. In response to boosting, titers were similar for mice of either group. IgG subclass endpoint titer measurements also demonstrate that the peptide CT E29H conjugate induces peptide-specific titers earlier and higher than those induced through immunization with a CRM₁₉₇/Aβ 1-7 peptide conjugate, and similar to those measured in mice immunized with a CT E29H adjuvanted Aβ 1-7 peptide/CRM₁₉₇ conjugate (Table 4). In response to the boosting immunization, titers measured in the sera of mice immunized twice with the Aβ 1-7 peptide/CT E29H conjugate were higher than those of mice immunized with the non-adjuvanted peptide CRM₁₉₇ conjugate.

TABLE 4
Aβ 1-42 PEPTIDE-SPECIFIC IGG SUBCLASS ENDPOINT TITERS
Groups of 5 Balb/c female mice were immunized twice, 4 weeks apart, with
the indicated conjugates. One group of mice also received CT E29H admixed
with the CRM₁₉₇ conjugate of the first seven amino acids of β amyloid
peptide. GeoMean endpoint titers +/− standard error are for sera
collected 4 weeks after primary immunization, and 2 weeks after
boosting immunization.

| | | Aβ 1-7/CT E29H | Aβ 1-7/CRM₁₉₇ | Aβ 1-7/CRM₁₉₇ + CT E29H |
|---|---|---|---|---|
| Week 4 | IgG1 | 1,804 ± 467 | 78 ± 26 | 1,479 ± 500 |
| | IgG2a | 653 ± 184 | * | 329 ± 175 |
| | IgG2b | ** | ** | ** |
| Week 6 | IgG1 | 4,919 ± 1,141 | 19,658 ± 16,706 | 198,278 ± 52,013 |
| | IgG2a | 1,452 ± 559 | *** | 17,116 ± 11,168 |
| | IgG2b | 754 ± 8 | *** | 1,824 ± 1,909 |

* titer not measurable at 1/75 dilution
** titer not measurable at 1/1000 dilution
*** titer not measurable at 1/500 dilution

### Example 8

### Mucosal (Intranasal) Immunogenicity Studies

Studies were also conducted with mice to evaluate the immunogenicity of Aβ 1-7 peptide/CT E29H conjugate when delivered via a mucosal route. In the following example, groups of mice were immunized with the indicated conjugate(s), delivered equally into each nares in a total volume of 10 ul, unless indicated otherwise. Mice were anaesthetized prior to nasal delivery of immunogens. For most studies, mice were immunized using a 2 week time interval between delivery, and were bled one day prior to immunization.

Groups of 10 Swiss Webster female mice, aged 7-9 weeks at the start of this study, were immunized with 5 ug of either Aβ 1-7 peptide/CT E29H conjugate or Aβ 1-7 peptide/CRM₁₉₇ conjugate in a volume of 10 ul on weeks 0, 2, and 4. Sera from weeks 2, 4, and 6 weeks post initial vaccination were analyzed for anti-Aβ 1-42 IgG, IgG1 and IgG2a titers. Nasal and vaginal washes were collected at week 6 and pooled for sample analysis of IgG and IgA titers. Results are presented for individual mice for IgG (Table 5) and IgG subclass titers (Table 6). Only 2 weeks after intranasal immunization, 5 of 10 mice receiving the Aβ 1-7 peptide/CT E29H conjugate had developed measurable peptide-specific serum IgG titers. None of the mice immunized with the CRM₁₉₇ conjugate of Aβ 1-7 had measurable titers, and even after 3 immunizations, several of the mice receiving this conjugate did not develop detectable serum IgG (Table 5). In contrast, all mice immunized with the Aβ 1-7 peptide/CT E29H conjugate developed serum IgG specific for Aβ 1-42 peptide within 2 weeks of the second immunization. Similarly, peptide-specific IgG1 and IgG2a titers were several fold higher in mice immunized with the Aβ 1-7 peptide/CT E29H conjugate than they were in mice immunized with Aβ 1-7/CRM₁₉₇ (Table 6).

TABLE 5
ANTI-Aβ 1-42 PEPTIDE IGG ENDPOINT TITERS IN
RESPONSE TO NASAL DELIVERY OF CONJUGATE VACCINE

| | Week 2 | Week 2 | Week 4 | Week 4 | Week 6 | Week 6 |
|---|---|---|---|---|---|---|
| Individual | Aβ1-7/CRM₁₉₇ | Aβ1-7/E29H | Aβ1-7/CRM₁₉₇ | Aβ1-7/E29H | Aβ1-7/CRM₁₉₇ | Aβ1-7/E29H |
| 1 | 50 | 50 | 50 | 281 | 50 | 2,787 |
| 2 | 50 | 645 | 50 | 8,387 | 279 | 45,347 |
| 3 | 50 | 50 | 50 | 591 | 50 | 9,633 |
| 4 | 50 | 50 | 50 | 1,885 | 50 | 21,295 |
| 5 | 50 | 50 | 50 | 949 | 50 | 1,446 |
| 6 | 50 | 614 | 50 | 28,157 | 91 | 29,240 |
| 7 | 50 | 301 | 50 | 91,708 | 50 | 10,317 |
| 8 | 50 | 3,734 | 50 | 59,694 | 50 | 163,627 |
| 9 | 50 | 50 | 50 | 23,244 | 1,989 | 10,650 |
| 10 | 50 | 931 | 586 | 84,680 | 1,028 | 396,913 |
| GeoMean | 50 | 205 | 64 | 7,724 | 23 | 19,468 |
| Std error | 50 | 104 | 16 | 5,333 | 55 | 10,526 |

TABLE 6
ANTI-Aβ 1-42 PEPTIDE IGG SUBCLASS
ENDPOINT TITERS IN RESPONSE TO NASAL
DELIVERY OF CONJUGATE VACCINE

| | |
|---|---|
| IgG1 | IgG2a |

| | Aβ1-7/ | Aβ1-7/ | Aβ1-7/ | Aβ1-7/ |
|---|---|---|---|---|
| Individual | CRM₁₉₇ | E29H | CRM₁₉₇ | E29H |
| 1 | 50 | 857 | 50 | 2,824 |
| 2 | 50 | 64,804 | 50 | 4,075 |
| 3 | 50 | 4,889 | 50 | 6,379 |
| 4 | 50 | 4,434 | 50 | 6,246 |
| 5 | 50 | 574 | 50 | 340 |
| 6 | 153 | 29,157 | 50 | 1,110 |
| 7 | 50 | 6,321 | 50 | 1,881 |
| 8 | 50 | 69,779 | 50 | 10,015 |
| 9 | 305 | 1,601 | 1,653 | 2,516 |
| 10 | 243 | 74,010 | 2,041 | 6,597 |
| GeoMean | 78 | 7,984 | 103 | 2,974 |
| Std error | 18 | 4,675 | 49 | 954 |

*Titers were determined after 3 immunizations from sera obtained 2 weeks after the final immunization (week 6).

Mucosal lavage IgG endpoint titers were determined from a pool of individual lavages (Table 7). Titers were determined after 3 immunizations from washes obtained from all animals (pooled) 2 weeks after the final immunization (week 6). Mucosal immunity was assessed using vaginal or nasal lavage. This was accomplished by instillation of 75 ul RPMI-10 into the vaginal vault of female mice using a 200 ul pipette, or by washing the nares of mice as described. The vault was washed by repeated delivery and removal of fluid, which was then added to 10 ul of FBS. These data demonstrate that peptide-specific IgG and IgA titers were only detected in mice immunized intranasally with the Aβ 1-7 peptide/CT E29H conjugate.

TABLE 7
MUCOSAL ANTI-Aβ 1-42 PEPTIDE IGG
AND IGA ENDPOINT TITERS IN RESPONSE TO
NASAL DELIVERY OF CONJUGATE VACCINE

| | |
|---|---|
| IgG | IgA |

| | Ab1-7/ | Ab1-7/ | Ab1-7/ | Ab1-7/ |
|---|---|---|---|---|
| | CRM | E29H | CRM | E29H |
| Vaginal Wash | 5 | 114 | 5 | 28 |
| Nasal Wash | 5 | 189 | 5 | 5 |

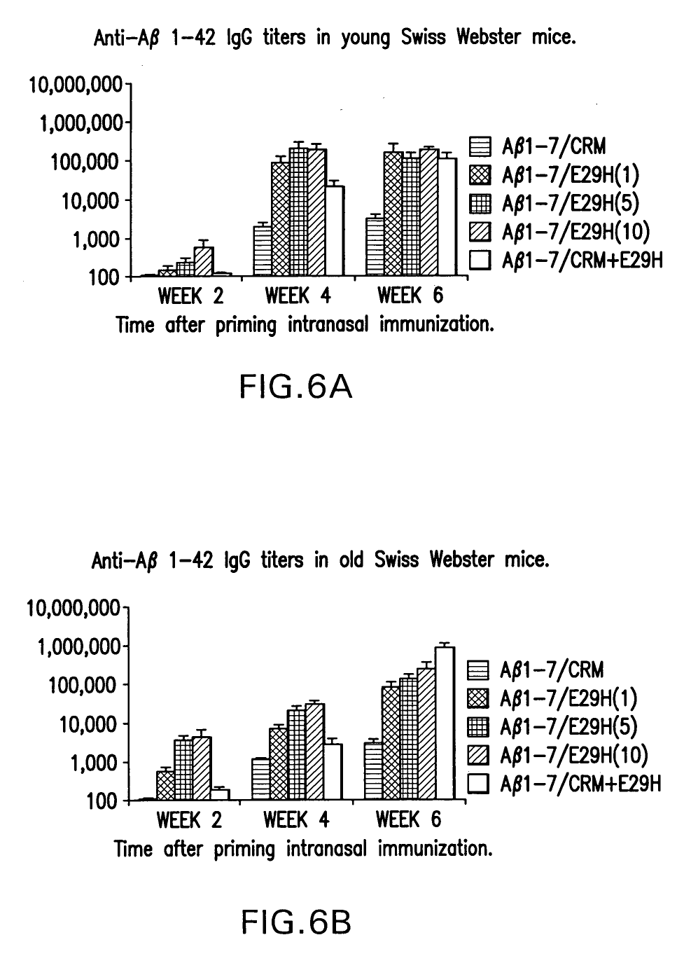

In a separate study, anti-Aβ 1-42 IgG endpoint titers from groups of 10 Swiss Webster female mice, aged 7-9 weeks at the time of initial immunization, were compared with those of 9 month old mice (FIG. 6). The data were collected from mice immunized by intranasal inoculation of 1, 5, or 10 ug doses of Aβ 1-7/CT E29H conjugate, or with 5 ug of Aβ 1-7/CRM₁₉₇ conjugate with or without 1 ug of CT E29H adjuvant. The anti-peptide antibody titers measured in the sera of mice were similar for the young and older mice. In neither age group, did mice respond to the peptide determinant in response to a single immunization with the non-adjuvanted Aβ 1-7/CRM₁₉₇ conjugate. At all time points, titers were generally 10-fold or less than those measured in mice immunized with any dose of the Aβ 1-7/CT E29H conjugate. Endpoint titers measured in mice immunized with Aβ 1-7/CT E29H conjugate were higher (weeks 2 and 4) or similar to (week 6) those measured in mice immunized with CT E29H adjuvanted Aβ 1-7/CRM₁₉₇ conjugate. Intranasal immunization with Aβ 1-7/CT E29H conjugate resulted in earlier detection and higher titers of peptide-specific IgG titers at a lower dose than induced through immunization with an Aβ 1-7/CRM₁₉₇ conjugate.

### Example 9

### Adjuvant Activity of Aβ 1-7/CT E29H Conjugate for Non-Conjugated Antigens/Epitopes

The findings described in the preceding confirmed that a peptide conjugate of CT E29H was more immunogenic than that same peptide conjugated to CRM₁₉₇. Those observations suggested that as a conjugate, CT E29H maintained its systemic and mucosal adjuvant activity, and helped in the induction of antibody titers specific for a small non-immunogenic peptide of 7 amino acids. To determine whether this assumption was true, another protein antigen was admixed with the Aβ 1-7/CT E29H conjugate, and mice were subcutaneously immunized. Sera of mice were bled at various time points after immunization and measured for antibody specific not only for the peptide, but for the immunizing protein. In the accompanying example, groups of 5 Swiss Webster female mice were immunized with Aβ 1-7/CT E29H conjugate together with a recombinantly expressed Neisseria gonorrhoeae pilin protein (International Application No. WO 00/49016). Mice were immunized at time 0, and boosted with the same 3 weeks later. Sera were collected for analysis at the initiation of the study, and the day prior to, and 2 weeks after the second immunization. The results show that in response to both immunizations, titers were higher in the mice immunized with the combination of the pilin and the Aβ 1-7/CT E29H conjugate, than with the Aβ 1-7/CRM₁₉₇ conjugate (Table 8). Anti-GC pilin IgG antibody endpoint titers were measured. Groups of 5 Swiss Webster mice were immunized as indicated on day 0 and boosted on week 3. Titers represent endpoint readings at an optical density cut off value of 0.1. Plates were coated with rGC pilin protein.

TABLE 8
ADJUVANT ACTIVITY ASSOCIATED WITH
Aβ 1-7/CT E29H CONJUGATE

antigen:

| | |
|---|---|
| Aβ 1-7/CRM + | Aβ 1-7/CT |
| rGCpilin | E29H + rGCpilin |

adjuvant:

| | |
|---|---|
| none | none |

anti-pilin IgG (week 3)

| | | | |
|---|---|---|---|
| individual | 1 | 29,229 | 77,385 |
| | 2 | 6,441 | 26,825 |
| | 3 | 10,170 | 48,390 |
| | 4 | 5,200 | 53,170 |
| | 5 | 5,202 | 77,535 |
| GeoMean | | 8,767 | 52,896 |
| Std Error | | 2849 | 10,311 |

anti-pilin IgG (week 5)

| | | | |
|---|---|---|---|
| individual | 1 | 828,232 | 934,497 |
| | 2 | 151,591 | 660,472 |
| | 3 | 790,923 | 1,899,793 |
| | 4 | 651,261 | 786,529 |
| | 5 | 301,228 | 959,441 |
| GeoMean | | 454,906 | 975,830 |
| Std Error | | 149,886 | 175,224 |

### Example 10

### Y-1 Adrenal Cell Assay for Wild-Type CT and Mutant CT Toxicity

Mutant CT polypeptides (e.g., E29H) were compared with wild-type CT for toxicity in the mouse Y-1 adrenal tumor cell assay. Y-1 adrenal cells (ATCC CCL-79) were seeded in 96-well flat-bottom plates at a concentration of 104 cells per well. Thereafter, three-fold serial dilutions of CT-CRMs were added to the tumor cells and incubated at 37° C. (5% CO₂) for 18 hours. The cells were then examined by light microscopy for evidence of toxicity (cell rounding). The endpoint titer is defined as the minimum concentration of toxin required to give greater than 50% cell rounding. The percent of residual toxicity was calculated using the endpoint titer of wild-type CT divided by the titer elicited by mutant CT multiplied by 100. Table 9 depicts the residual toxicity of several purified antigen-mutant CT conjugates tested in the Y-1 adrenal cell assay.

TABLE 9
E29H CONJUGATES DEMONSTRATE REDUCED TOXICITY

| Test Sample | Percent Toxicity |
|---|---|
| Aβ 1-7/E29H | 0.083 |
| *GBSIII/E29H | 0.100 |
| *NMB-LOS/E29H | 0.040 |
| E29H | 0.370 |

*GBSIII is Group B Strep antigen
*NMB is either a 4.5 kDa wildtype lipooligosaccharide (LOS) or a 3.2 kDa truncated LOS.

### Example 11

### CT E29H as a Lipooligosaccharide (LOS) Carrier

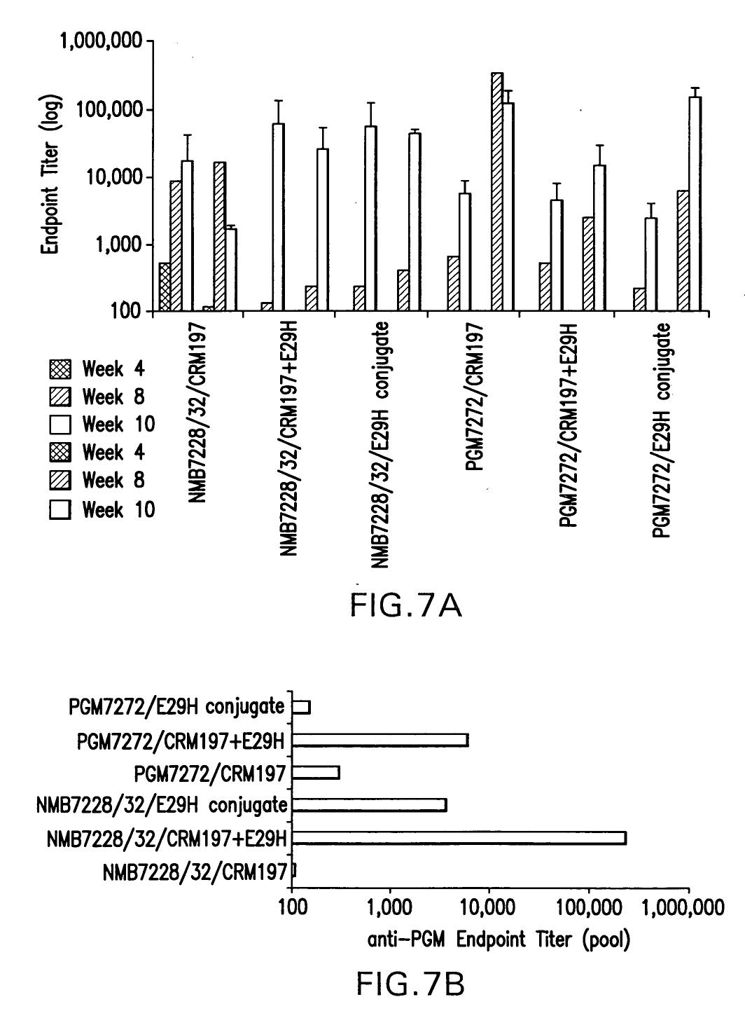

Two conjugates of Meningococcal LOS were prepared using E29H as a carrier: NMB7228/32, a 4.5 kDa wild type LOS expressing outer and inner core saccharides, and NMBPGM7232, a 3.2 kDa truncated LOS expressing only inner core saccharide structures. LOS was de-O-acylated by mild alkaline treatment and conjugated to E29H using succinimidyl 3-(2-pyridyldithio)propionate (SPDP) chemistry. Bromoacetylation of E29H with N-Succinimidyl Bromoacetate was required for LOS crosslinking. To test for immunogenicity, groups of five Swiss Webster female mice were immunized subcutaneously with 5 ug (total protein) of the indicated conjugates, with or without a supplemental E29H adjuvant (5 ug), at weeks 0, 4 and 8. Sera from two separate studies were collected for antibody analysis at the indicated time points, and assayed against both the wild type and the truncated LOS. FIG. 7A and 7B demonstrate that E29H acts as a carrier for LOS. In FIG. 7A, titers are shown as measured from pools of sera collected at weeks 4 and 8, and as a GeoMean of individuals (±SE) for week 10, and in FIG. 7B, titers are shown as measured from pools of sera collected at week 10.

In separate studies (data not shown), immunization of Swiss Webster mice with native LOS conjugated to CRM₁₉₇ and adjuvanted with E29H, or conjugated to E29H directly, induced antibody titers to native LOS that were several fold higher than induced through immunization with a non-adjuvanted LOS/CRM₁₉₇ conjugate alone. The E29H conjugate demonstrated modest adjuvanting activity.

### Example 12

### E29H Conjugates of GBSIII Demonstrate Similar or Enhanced Antibody Responses When Compared With a Conjugate of GBSIII/CRM₁₉₇

Group B Strep antigen (GBSIII) was successfully and repeatedly conjugated to E29H by reductive amination. Carbohydrate to protein ratio during conjugation was 1:1. Polysaccharide was oxidized in acetate buffer, and was lyophilized prior to conjugation. E29H was added to the lyophilized polysaccharide along with the conjugation buffer to solubilize the preparation prior to characterization and immunization studies.

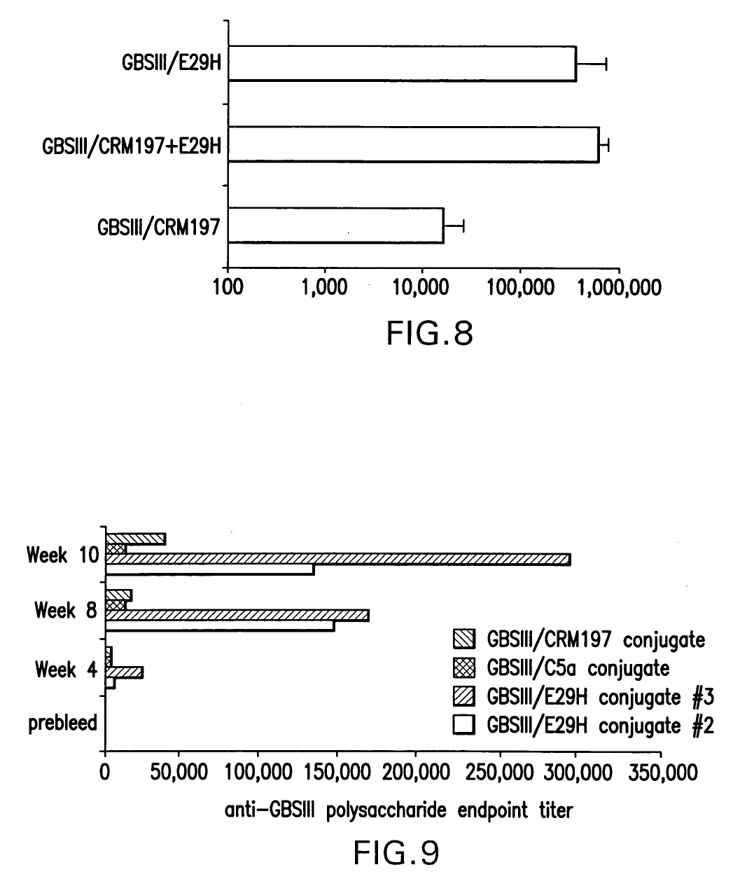

Titers measured from mice bled after three immunizations with the GBSIII/E29H conjugate were similar to those of mice immunized with a GBSIII/CRM₁₉₇ conjugate adjuvanted with 5 ug E29H. Titers were approximately 10-fold higher than those induced in mice immunized with non-adjuvanted CRM₁₉₇ conjugated GBSIII (FIG. 8).

### Example 13

### GBSIII/E29H Conjugates Demonstrate Similar or Enhanced Antibody Responses When Compared With GBSIII Conjugates of CRM₁₉₇ or C5A

Two conjugates of GBSIII/E29H were evaluated in a murine immunogenicity study with a GBSIII/CRM₁₉₇ and GBSIII/C5a conjugate. C5s is a 74 amino acid glycopeptide cleaved from the fifth component (C5) of complement, which acts as a chemical signal to stimulate the inflammatory response in mammals. In addition, C5a is a substrate for the streptococcal C5a peptidase. Groups of 5 Swiss Webster female mice were immunized subcutaneously with 5 ug (total protein) of the indicated conjugates without supplemental adjuvant, at weeks 0, 4 and 6. Sera were collected as pools for measurement of GBSIII polysaccharide specific antibodies at the indicated time points.

E29H acts as a carrier for GBSIII, and appears to adjuvant the response specific for the conjugated polysaccharide. As a carrier protein, E29H appears more effective in the absence of exogenous adjuvant for the induction of GBSIII specific IgG antibody than CRM₁₉₇ or C5a (FIG. 9).

### Example 14

### An E29H Conjugate Demonstrates Adjuvant Properties Specific for an Admixed, Non-Conjugate Associated Antigen

Recombinant GC pilin protein was mixed with either CRM₁₉₇ or E29H conjugates of Aβ 1-7 peptide. Groups of 5 Swiss Webster female mice were immunized with 5 ug conjugate (total protein) and 10 ug of the pilin protein. Mice were immunized subcutaneously on weeks 0 and 3. Individual sera were collected and measured for peptide specific IgG antibody titers three weeks after initial immunization, and 2 weeks after boosting immunization.

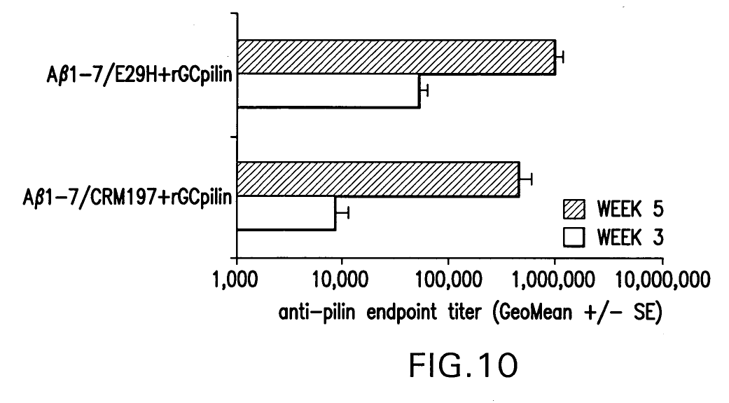

The E29H conjugate is an effective adjuvant for a “bystander” antigen. Even at week 3, titers mice immunized with the E29H conjugate were more than 6-fold those of mice immunized with the CRM₁₉₇ conjugate (FIG. 10).

### Example 15

### Preparation and Purification of a LOS-CT E29H Conjugate

Dephosphorylated and O-deacylated recombinant Chlamydial LOS (rChlamydial LOS; 2.3 mg) was dissolved in 1.12 ml of CT E29H solution (2.05 mg/ml). The pH of the solution was adjusted to 8.9 by adding 150 ul of 0.05 M sodium borate, pH 9.25. Sodium cyanoborohydrate was added in 10-fold excess and reaction mixture was kept for eight hours at ambient temperature and then for four days at 37° C. in an incubator. The reaction yielding the conjugate was stopped by addition of 76 ug of sodium borohydride (7.6 ul of 10 mg/ml solution) and incubated for one hour at ambient temperature.

The rChlamydial LOS-CTE29H conjugate was then purified on a Sephacryl S300 (1.5×90 cm) column eluted with 0.9% NaCl. The chromatography was monitored by differential refractometer and by absorbance at 280 nm. The collected fractions were analyzed for the presence of rChlamydial LOS by thiobarbituric acid (TBA) assay and protein by Bradford assay. TBA is an assay for the colorimetric identification of the sugar KDO (2-keto-3-deoxy-manno-octonic acid) (Brade et al., Differential determination of the 3-Deoxy-D-mannooctulosonic acid residues in lipopolysaccharides of Salmonella minnesota rough mutants. Eur. J. Biochem. 131, 195-200 (1983)).

The fractions containing the conjugate were combined and concentrated to 1 mL on Amicon XY 60 membrane. The rChlamydial LOS-CTE29H conjugate was analyzed for LOS concentration by TBA assay using dephosphorylated O-deacylated rChlamydial LOS as the standard, and for protein concentration by Bradford assay using BSA as a standard.

### REFERENCES

European Patent Application No. EP 125,023
European Patent Application No. EP 171,496
European Patent Application No. EP 173,494
European Patent Application No. EP 184,187
European Patent Application No. 449,958.
International Application No. WO 00/18434
International Application No. WO 86/01533
International Application No. WO 91/17271
International Application No. WO 92/01047
International Application No. WO 92/09690
International Application No. WO 92/15679
International Application No. WO 92/18619
International Application No. WO 92/19265
International Application No. WO 92/20791
International Application No. WO 93/01288
International Application No. WO 93/13202
International Application No. WO 93/13302
International Application No. WO 98/42375
U.S. Pat. No. 4,196,265
U.S. Pat. No. 4,522,811
U.S. Pat. No. 4,554,101
U.S. Pat. No. 4,683,202
U.S. Pat. No. 4,816,567
U.S. Pat. No. 4,912,094
U.S. Pat. No. 4,992,463
U.S. Pat. No. 5,057,540
U.S. Pat. No. 5,078,996
U.S. Pat. No. 5,168,062
U.S. Pat. No. 5,223,409
U.S. Pat. No. 5,424,334
U.S. Pat. No. 5,552,431
U.S. Pat. No. 5,580,859
U.S. Pat. No. 5,593,972
U.S. Pat. No. 5,723,127
U.S. Pat. No. 6,113,918
U.S. Pat. No. 6,127,170
U.S. Pat. No. 6,168,918
U.S. Pat. No. 6,207,646
Asiam and Dent, “Bioconjugation: Protein Coupling Techniques for the Biomedical Sciences,” Macmillan Reference Ltd., London, England, 1998.
Amann et al., Gene 69:301-315, 1988.
Ausubel et al., Current Protocols in Molecular Biology, eds., John Wiley & Sons, Inc., sections 2.10 and 6.3-6.4, 1995.
Brandtzaeg, “Immune Functions of Human Nasal Mucosa and Tonsils in Health and Disease”, page 28 et seq. In Immunology of the Lung and Upper Respiratory Tract (Bienenstock, J., Ed., McGraw-Hill, New York, N.Y. (1984)).
Brandtzaeg and Baklein, Scand. J. Gastroenterol., 11 :(Suppl. 36), 1-45, 1976.
Burns, et al., Science, 272:104-107, 1996.
Crabbe, et aL, J. Exptl. Med., 130:723-744, 1969.
Craig and Cebra, J. Exptl. Med., 134:188-200, 1971.
Crawford, et al., J. Virology, 68:5945-5952, 1994.
Cuatrecasas, Biochemistry, 12:3558-3566, 1973.
Elson, C. O., and Ealding, W., J. Immunol., 132:2736-2741, 1984.
Frohman et al., Proc. NatI. Acad. Sci. USA 85, 8998-9002, 1988.
Gill and Meren, Proc. Natl. Acad. Sci., USA, 75:3050-3054, 1978.
Gill, and Meren Proc. Natl. Acad. Sci., USA, 75:3050-3054, 1978.
Gill, Biochemistry, 15:1242-1248, 1976.
Guidry, et al., Infect. Immun., 65:4943-4950, 1997.
Harlow and Lane, “Antibodies: A Laboratory Manual,” Cold Spring Harbor Laboratory Press, Cold Spring Harbor, N.Y., 1988
Hansen, et al., Infect. Immun., 56:182-190, 1988.
Hanson, L. A., Intl. Arch. Allergy Appl. Immunol., 18:241-267 (1961).
Hu, et al., Infect Immun., 60:2657-2666, 1992.
Ishida, et al., J. Clin. Microbiol., 34:1694-1700, 1996.
Jobling and Holmes, Infect Immun., 60:4915-4924, 1992.
Jobling and Holmes, Mol. Microbiol., 5:1755-1767, 1991.
Karasic, et al, Ped. Inf. Dis. J., 8:(Suppl.), S62-65, 1988.
Kassis et al, J. Biol. Chem, 257:12148-12152, 1982.
Klipstein et al., “Vaccine for Enterotoxigenic Escherichia coli Based on Synthetic Heat-Stable Toxin Cross-Linked to the B Subunit of Heat Labile Toxin,” Journal of Infect. Diseases, 147:318-326, 1983.
Kunkel, Proc. Natl. Acad. Sci., USA, 82:488-492, 1985.
Kyte and Doolittle, J. Mol. Biol., 157:105-132, 1982.
Laemmll, Nature (London), 227:680-685, 1970.
Lee, et al., Gastroenterology, 112:1386-1397, 1997.
Maneval, et al, J. Tissue Cult. Methods, 6:85-90, 1981.
McNeal and Ward, Virology, 211:474-480, 1995.
Mekalanos et at, Nature, 306:551-557, 1983.
Mekalanos, et al., J. Biol. Chem., 254:5855-5861, 1979.
Mestecky, et al., J. Clin. Invest., 61:731-737, 1978.
O'Neal, et al., J. Virology, 71:8707-8717, 1997.
O'Neal, et al., J. Virology, 72:3390-3393, 1998.
Pachuk, et a., Curr. Topics Microbiol. Immunol, 59:1024-1031, 1997.
Rudin, et al., Infect. Immun., 66:3390-3396, 1998.
Sambrook et al., “Molecular Cloning: A Laboratory Manual” 2nd, ed, Cold Spring Harbor Laboratory, Cold Spring Harbor Laboratory Press, Cold Spring Harbor, N.Y., 1989.
Sanger, et al., Proc. Natl. Acad. Sci., USA, 74:5463-5467, 1977.
Smith and Johnson, Gene 67:31-40, 1988.
Snider, et al., J. Immunol. 153:647-657, 1994.
Solari and Kraehenbuhl, Immunol. Today, 6:17-20, 1985.
Studier et al “Gene Expression Technology” Methods in Enzymology 185, 60-89, 1990.
Tamura, et al., Vaccine, 12:1238-1240, 1994.
Tartof and Hobbs, Focus, 9:12, 1987.
Tomasi, T. B., and Zigelbaum, S., J. Clin. Invest, 42:1552-1560, 1963.
Tomasi, T. B., et al, J. Exptl. Med., 121:101-124, 1965.
Van der Akker, et al., Structure, 4:665-678, 1996.
Walsh, et al., Infect. Immun., 43:756-758, 1984.
Welsh et al., “ADP-Ribosylation Factors: A Family of Guanine Nucleotide-Binding Proteins that Activate Cholera Toxin and Regulate Vesicular Transport”, pages 257-280 in Handbook of Natural Toxins: Bacterial Toxins and Virulence Factors in Disease, Vol. 8 (Moss, J., et al., Eds., Marcel Dekker, Inc., New York, N.Y., 1995.
Maneval, et al., J. Tissue Cult. Methods, 6:85-90, 1981.

What is claimed is:

1. An immunogenic composition comprising a cholera holotoxin (CT) and an antigen covalently associated with the CT, wherein the CT comprises an A subunit (CT-A ) having a mutation of at least amino acid residue 29 of SEQ ID NO:2, wherein the mutation is not an aspartic acid, wherein the CT increases immunogenicity of the antigen.

2. The composition of claim 1, wherein the CT is further defined as having reduced toxicity relative to a CT comprising a wild-type CT-A.

3. The composition of claim 1, wherein the CT-A is encoded by a polynucleotide comprising a nucleic acid sequence of SEQ ID NO:1 or a degenerate variant thereof, wherein the nucleotide sequence has a genetic modification of at least codon 29 of SEQ ID NO:1.

4. The composition of claim 1, wherein residue 29 of SEQ ID NO:2 is an amino acid selected from the group consisting of Ala, Cys, Phe, Gly, His, Ile, Lys, Leu, Met, Asn, Pro, GIn, Arg, Ser, Thr, Val, Trp and Tyr.

5. The composition of claim 4, wherein residue 29 is a His residue.

6. The composition of claim 1, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

7. The composition of claim 1, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

8. The composition of claim 1, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

9. The composition of claim 1, further comprising one or more adjuvants.

10. The composition of claim 9, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1α, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor a and tumor necrosis factor β.

11. The composition of claim 1, further comprising a pharmaceutically acceptable carrier.

12. An immunogenic composition comprising a CT and an antigen covalently associated with the CT, wherein the CT comprises one or more mutations in the CT-A, wherein the CT increases immunogenicity of the antigen.

13. The composition of claim 12, wherein the CT is further defined as having reduced toxicity relative to a CT comprising a wild-type CT-A.

14. The composition of claim 12, wherein the CT-A comprises an amino acid sequence of SEQ ID NO:2.

15. The composition of claim 12, wherein the CT-A is encoded by a polynucleotide comprising a nucleic acid sequence of SEQ ID NO:1 or a degenerate variant thereof.

16. The composition of claim 14, wherein the one or more mutations are selected from the group consisting of Arg-7, Asp-9, Arg-11, Ile-16, Arg-25, Glu-29, Trp-30, His-44, Val-53, Ser-63, Ser-68, His-70, Val-72, Val-97, Tyr-104, Pro-106, Ser-109, Glu-112 and Arg-192, wherein Glu-29 is not mutated to Asp-29.

17. The composition of claim 16, wherein one mutation is at Glu-29, wherein the mutation at Glu-29 is not Asp-29.

18. The composition of claim 17, wherein Glu-29 is mutated to a His-29 residue.

19. The composition of claim 16, wherein one or more mutations is a double mutation at Ile-16 and Ser-68.

20. The composition of claim 16, wherein one or more mutations is a double mutation at Ser-68 and Val-72.

21. The composition of claim 19, wherein Ile-16 is mutated to Ala-16 and Ser-68 is mutated to Tyr-68.

22. The composition of claim 20, wherein Ser-68 is mutated to Tyr-68 and Val-72 is mutated to Tyr-72.

23. The composition of claim 14, wherein one or more mutations is an amino acid insertion at amino acid position 49.

24. The composition of claim 14, wherein one or more mutations is an amino acid insertion at amino acid position 36 and an insertion at amino acid position 37.

25. The composition of claim 14, wherein one or more mutations is an amino acid substitution at amino acid position 30, an amino acid insertion at amino acid position 31 and an insertion at amino acid position 32.

26. The composition of claim 23, wherein a histidine amino acid is inserted at amino acid position 49 between the wild-type amino acid positions 48 and 49.

27. The composition of claim 24, wherein the amino acids glycine and proline are inserted in the amino acid positions 36 and 37 between the wild-type amino acid positions 34 and 35.

28. The composition of claim 25, wherein the mutation at amino acid position 30 is a tryptophan and alanine and a histidine are inserted in the amino acid positions 31 and 32 between the wild-type amino acid positions 30 and 31.

29. The composition of claim 12, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

30. The composition of claim 12, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

31. The composition of claim 12, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

32. The composition of claim 12, further comprising one or more adjuvants.

33. The composition of claim 32, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor a and tumor necrosis factor β.

34. The composition of claim 12, further comprising a pharmaceutically acceptable carrier.

35. An immunogenic composition comprising an Escherichia coli heat labile toxin (LT) and an antigen covalently associated with the LT, wherein the LT increases immunogenicity of the antigen.

36. The composition of claim 35, wherein the LT is further defined as having one or more mutations in the LT-A subunit.

37. The composition of claim 36, wherein the one or more mutations are selected from the group consisting of Val-53, Ser-63, Ala-72, Val-97, Tyr-104, Pro-106 and Arg-192.

38. The composition of claim 37, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

39. The composition of claim 37, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

40. The composition of claim 37, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

41. The composition of claim 37, further comprising one or more adjuvants.

42. The composition of claim 41, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor a and tumor necrosis factor β.

43. The composition of claim 37, further comprising a pharmaceutically acceptable carrier.

44. An immunogenic composition comprising a pertussis toxin (PT) and an antigen covalently associated with the PT, wherein the PT increases immunogenicity of the antigen.

45. The composition of claim 44, wherein the PT is further defined as having one or more mutations in the PT-A subunit.

46. The composition of claim 44, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

47. The composition of claim 44, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

48. The composition of claim 44, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

49. The composition of claim 44, further comprising one or more adjuvants.

50. The composition of claim 49, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor a and tumor necrosis factor β.

51. The composition of claim 44, further comprising a pharmaceutically acceptable carrier.

52. A method of immunizing a mammalian host comprising administering to the host an immunogenic amount of a composition comprising a cholera holotoxin (CT) and an antigen covalently associated with the CT, wherein the CT comprises an A subunit (CT-A ) having a mutation of at least amino acid residue 29 of SEQ ID NO:2, wherein the mutation is not an aspartic acid, wherein the CT increases immunogenicity of the antigen.

53. The method of claim 52, wherein the CT is further defined as having reduced toxicity relative to a CT comprising a wild-type CT-A.

54. The method of claim 52, wherein the CT-A is encoded by a polynucleotide comprising a nucleic acid sequence of SEQ ID NO:1 or a degenerate variant thereof, wherein the nucleotide sequence has a genetic modification of at least codon 29 of SEQ ID NO:1.

55. The method of claim 52, wherein residue 29 of SEQ ID NO:2 is an amino acid selected from the group consisting of Ala, Cys, Phe, Gly, His, Ile, Lys, Leu, Met, Asn, Pro, Gin, Arg, Ser, Thr, Val, Trp and Tyr.

56. The method of claim 55, wherein residue 29 is a His residue.

57. The method of claim 52, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

58. The method of claim 52, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

59. The method of claim 52, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

60. The method of claim 52, further comprising one or more adjuvants.

61. The method of claim 60, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor a and tumor necrosis factor β.

62. The method of claim 52, further comprising a pharmaceutically acceptable carrier.

63. A method of immunizing a mammalian host comprising administering to the host an immunogenic amount of a composition comprising a CT and an antigen covalently associated with the CT, wherein the CT comprises one or more mutations in the CT-A, wherein the CT increases immunogenicity of the antigen.

64. The method of claim 63, wherein the CT is further defined as having reduced toxicity relative to a CT comprising a wild-type CT-A.

65. The method of claim 63, wherein the CT-A comprises an amino acid sequence of SEQ ID NO:2.

66. The method of claim 63, wherein the CT-A is encoded. by a polynucleotide comprising a nucleic acid sequence of SEQ ID NO:1 or a degenerate variant thereof.

67. The method of claim 65, wherein the one or more mutations are selected from the group consisting of Arg-7, Asp-9, Arg-11, Ile-16, Arg-25, Glu-29, Trp-30, His-44, Val-53, Ser-63, Ser-68, His-70, Val-72, Val-97, Tyr-104, Pro-106, Ser-109, Glu-112 and Arg-192, wherein Glu-29 is not mutated to Asp-29.

68. The method of claim 67, wherein one mutation is at Glu-29, wherein the mutation at Glu-29 is not Asp-29.

69. The method of claim 68, wherein Glu-29 is mutated to a His-29 residue.

70. The method of claim 67, wherein one or more mutations is a double mutation at Ile-16 and Ser-68.

71. The method of claim 67, wherein one or more mutations is a double mutation at Ser-68 and Val-72.

72. The method of claim 70, wherein Ile-16 is mutated to Ala-16 and Ser-68 is mutated to Tyr-68.

73. The method of claim 71, wherein Ser-68 is mutated to Tyr-68 and Val-72 is mutated to Tyr-72.

74. The method of claim 65, wherein one or more mutations is an amino acid insertion at amino acid position 49.

75. The method of claim 65, wherein one or more mutations is an amino acid insertion at amino acid position 36 and an insertion at amino acid position 37.

76. The method of claim 65, wherein one or more mutations is an amino acid substitution at amino acid position 30, an amino acid insertion at amino acid position 31 and an insertion at amino acid position 32.

77. The method of claim 74, wherein a histidine amino acid is inserted at amino acid position 49 between the wild-type amino acid positions 48 and 49.

78. The method of claim 75, wherein the amino acids glycine and proline are inserted in the amino acid positions 36 and 37 between the wild-type amino acid positions 34 and 35.

79. The method of claim 76, wherein the mutation at amino acid position 30 is a tryptophan and alanine and a histidine are inserted in the amino acid positions 31 and 32 between the wild-type amino acid positions 30 and 31.

80. The method of claim 63, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

81. The method of claim 63, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

82. The method of claim 63, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

83. The method of claim 63, further comprising one or more adjuvants.

84. The method of claim 83, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor a and tumor necrosis factor β.

85. The method of claim 63, further comprising a pharmaceutically acceptable carrier.

86. A method of immunizing a mammalian host comprising administering to the host an immunogenic amount of a composition comprising an Escherichia coli heat labile toxin (LT) and an antigen covalently associated with the LT, wherein the LT increases immunogenicity of the antigen.

87. The method of claim 86, wherein the LT is further defined as having one or. more mutations in the LT-A subunit.

88. The method of claim 87, wherein the one or more mutations are selected from the group consisting of Val-53, Ser-63, Ala-72, Val-97, Tyr-104, Pro-106 and Arg-192.

89. The method of claim 88, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

90. The method of claim 88, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

91. The method of claim 88, further comprising one or more additional non-covalently associated antigens selected from the group -consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

92. The method of claim 88, further comprising one or more adjuvants.

93. The method of claim 92, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7,
IL-18, interferon-α, interferon-β, interferon-γ, granulocyte colony stimulating factor, tumor necrosis factor a and tumor necrosis factor β.

94. The method of claim 88, further comprising a pharmaceutically acceptable carrier.

95. A method of immunizing a mammalian host comprising administering to the host an immunogenic amount of a composition comprising a pertussis toxin (PT) and an antigen covalently associated with the PT, wherein the PT increases immunogenicity of the antigen.

96. The composition of claim 95, wherein the antigen is selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

97. The composition of claim 95, further comprising one or more additional covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

98. The composition of claim 95, further comprising one or more additional non-covalently associated antigens selected from the group consisting of a polypeptide, a polypeptide fragment, a carbohydrate, an oligosaccharide, a lipid, a lipooligosaccharide, a polysaccharide, an oligosaccharide-protein conjugate, a polysaccharide-protein conjugate, a peptide-protein conjugate, an oligosaccharide-peptide conjugate, a polysaccharide-peptide conjugate, a protein-protein conjugate, a lipooligosaccharide-protein conjugate and a polysaccharide-protein conjugate.

99. The composition of claim 95, further comprising one or more adjuvants.

100. The composition of claim 99, wherein one or more adjuvants are selected from the group consisting of GM-CSF, 529SE, IL-12, aluminum phosphate, aluminum hydroxide, Mycobacterium tuberculosis, Bordetella pertussis, bacterial lipopolysaccharides, aminoalkyl glucosamine phosphate compounds, MPL (3-O-deacylated monophosphoryl lipid A), a polypeptide, Quil A, QS-21, a pertussis toxin (PT), an E. coli heat-labile toxin (LT), IL-1α, IL-1β, IL-2, IL-4, IL-5, IL-6, IL-7, IL-8, IL-10, IL-13, IL-14, IL-15, IL-16, IL-7, IL-18, interferon-α, interferon-β, interferon-γ, granulocyte colony stimulating factor, tumor necrosis factor α and tumor necrosis factor β.

101. The composition of claim 95, further comprising a pharmaceutically acceptable carrier.


SEQUENCE LISTING (8 sequences)

SEQ ID NO: 1 - DNA, 720 bp, Vibrio cholerae
aatgatgataagttatatcgggcagattctagacctcctgatgaaataaagcagtcaggtggtcttatgccaagaggacagagtcactactttgaccgaggtactcaaatgaatatcaacctttatgatcatgcaagaggaactcagacgggatttgttaggcacgatgatggatatgtttccacctcaattagtttgagaagtgcccacttagtgggtcaaactatattgtctggtcattctacttattatatatatgttatagccactgcacccaacatgtttaacgttaatgatgtattaggggcatacagtcctcatccagatgaacaagaagtttctgctttaggtgggattccatactcccaaatatatggatggtatcgagttcattttggggtgcttgatgaacaattacatcgtaataggggctacagagatagatattacagtaacttagatattgctccagcagcagatggttatggattggcaggtttccctccggagcatagagcttggagggaagagccgtggattcatcatgcaccgccgggttgtgggaatgctccaagatcatcgatgagtaatacttgcgatgaaaaaacccaaagtctaggtgtaaaattccttgacgaataccaatctaaagttaaaagacaaatattttcaggctatcaatctgatattgatacacataatagaattaaggatgaatta

SEQ ID NO: 2 - protein, 240 aa, Vibrio cholerae
NDDKLYRADSRPPDEIKQSGGLMPRGQSEYFDRGTQMNINLYDHARGTQTGFVRHDDGYVSTSISLRSAHLVGQTILSGHSTYYIYVIATAPNMFNVNDVLGAYSPHPDEQEVSALGGIPYSQIYGWYRVHFGVLDEQLHRNRGYRDRYYSNLDIAPAADGYGLAGFPPEHRAWREEPWIHHAPPGCGNAPRSSMSNTCDEKTQSLGVKFLDEYQSKVKRQIFSGYQSDIDTHNRIKDEL

SEQ ID NO: 3 - DNA, 20 bp, Artificial
  Synthetic oligo
aagttatataaggcagattc

SEQ ID NO: 4 - DNA, 18 bp, Artificial
  Synthetic nucleotide sequence
cagattctaaacctcctg

SEQ ID NO: 5 - DNA, 22 bp, Artificial
  synthetic oligo
gacagagtnagtactttgaccg

SEQ ID NO: 6 - DNA, 22 bp, Artificial
  synthetic oligo
cagatgancaagangtttctgc

SEQ ID NO: 7 - DNA, 22 bp, Artificial
  synthetic oligo
cagatgancaagangtttctgc

SEQ ID NO: 8 - protein, 7 aa, Homo sapiens
DAEFRHD